SEQUENCE LISTING (3 sequences)

SEQ ID NO: 1 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 2 - protein, 870 aa, Homo sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

SEQ ID NO: 3 - protein, 669 aa, Homo sapiens
MALGLQRARSTTELRKEKSRDAARSRRSQETEVLYQLAHTLPFARGVSAHLDKASIMRLTISYLRMHRLCAAGEWNQVGAGGEPLDACYLKALEGFVMVLTAEGDMAYLSENVSKHLGLSQLELIGHSIFDFIHPCDQEELQDALTPQQTLSRRKVEAPTERCFSLRMKSTLTSRGRTLNLKAATWKVLNCSGHMRAYKPPAQTSPAGSPDSEPPLQCLVLICEAIPHPGSLEPPLGRGAFLSRHSLDMKFTYCDDRIAEVAGYSPDDLIGCSAYEYIHALDSDAVSKSIHTLLSKGQAVTGQYRFLARSGGYLWTQTQATVVSGGRGPQSESIVCVHFLISQVEETGVVLSLEQTEQHSRRPIQRGAPSQKDTPNPGDSLDTPGPRILAFLHPPSLSEAALAADPRRFCSPDLRRLLGPILDGASVAATPSTPLATRHPQSPLSADLPDELPVGTENVHRLFTSGKDTEAVETDLDIAQDADALDLEMLAPYISMDDDFQLNASEQLPRAYHRPLGAVPRPRARSFHGLSPPALEPSLLPRWGSDPRLSCSSPSRGDPSASSPMAGARKRTLAQSSEDEDEGVELLGVRPPKRSPSPEHENFLLFPLSLSFLLTGGPAPGSLQDPSTPLLNLNEPLGLGPSLLSPYSDEDTTQPGGPFQPRAGSAQAD

(19) United States
(12) Patent Application Publication
(10) Pub. No.: US 2022/0347167 A1
(43) Pub. Date: Nov. 3, 2022

(54) A NEW TREATMENT FOR MEIBOMIAN GLAND DYSFUNCTION

(71) Applicant: The Schepens Eye Research Institute, Inc., Boston, MA (US)

(72) Inventor: Yang Liu, Winchester, MA (US)

(21) Appl. No.: 17/766,029

(22) PCT Filed: Oct. 2, 2020
(86) PCT No.: PCT/US2020/054091

§ 371 Date: Apr. 1, 2022

Related U.S. Application Data
(60) Provisional application No. 62/911,228, filed on Oct. 5, 2019.

(51) Int. Cl. A61K 31/47; A61K 31/22; A61K 31/16; A61K 33/24; A61K 9/00

(52) U.S. Cl. CPC A61K 31/47; A61K 31/22; A61K 31/16; A61K 33/24; A61K 9/0051

(57) ABSTRACT

The invention provides compositions and methods for reducing oxygen concentration in the environment of or promoting the expression or function of at least one hypoxia inducible factor (HIF) in one or more dysfunctional meibomian glands (MGs), utilizing a low oxygen mimetic agent capable of promoting the expression or function of at least one HIF and mimicking a low oxygen environment, or a combination thereof, to improve MG functions.

### PRIORITY INFORMATION

The present application is a U.S. National Stage Entry of the International Patent Application No. PCT/US2020/054091 filed on Oct. 2, 2020, which claims priority to U.S. Provisional Patent Application No. 62/911,228, filed Oct. 5, 2019, the content of which is incorporated herein by references in their entirety.

### STATEMENT AS TO FEDERALLY SPONSORED RESEARCH

This invention was made with government support under 821028653 and P30EY003790, awarded by National Institutes of Health. The government has certain rights in the invention.

### INCORPORATION OF THE SEQUENCE LISTING

The material in the accompanying Sequence Listing is hereby incorporated by reference into this application. The accompanying Sequence Listing text file, named 036770_586001WO_Sequence_Listing_ST25_ST25.txt, was created on Oct. 2, 2020 and is 24 KB.

### FIELD OF THE DISCLOSURE

The present disclosure generally relates to methods for treatment of various dysfunctions such as a meibomian gland dysfunction (MGD), dry eye disease, and obesity.

### BACKGROUND

Meibomian glands (MGs) are large lipid-producing glands located in the eyelids. Their oily secretion (i.e. meibum) keeps eyes moist and plays an essential role in maintaining the health of the ocular surface. In contrast, meibomian gland dysfunction (MGD), and the consequent meibum deficiency, destabilize the tear film and increase tear osmolarity and evaporation. At present, the LipiFlow thermal pulsation system is the only FDA-approved treatment or device for MGD. There is a significant need for innovative therapeutic strategies for MGD and related ocular diseases or disorders.

### SUMMARY

The invention provides methods of reducing the severity or treating an optical disease or disorder associated with reduced meibomian gland (MG) function in a subject by locally administering to an eye tissue of the subject a composition comprising a low oxygen mimetic compound. By “low oxygen” is meant less than 5% oxygen in a bodily tissue or fluid. For example, the tissue is an ocular tissue such as MG or a bodily fluid such as blood or serum. Treatment in this manner leads to an improved clinical condition of the subject, e.g., reduced severity or treatment of said disease or disorder comprises increased tear secretion, increased tear volume, prolonged tear film break up time, improved tear osmolarity, improved gland expressibility, improved gland expressed oil quality and volume, increased thickness of tear film lipid layer, decreased ocular surface staining, decreased inflammation on the ocular surface, and/or alleviated ocular discomfort feeling of patients. For example, the low oxygen mimetic compound comprises a hypoxia inducible factor (HIF) prolyl hydroxlase inhibitor (HIF-PHI) such as Roxadustat, Daprodustat, Molidustat, Vadadustat, or Desitustat. Alternatively, the composition comprises dimethyloxalyglycine, desferrioxamine, or cobalt (II) chloride (CoCl₂).

In preferred embodiments, the composition in the form of an eye drop or eye ointment, e.g., the composition is administered at a concentration of 0.001-100 mg/ml. For example, the concentration comprises 0.01-100 mg/ml, 10-100 μg/ml, or 10-50 μg/ml. In an example, the concentration is about 20 μg/ml. Eye drops or ointments are administered once, twice, thrice, 4 times, 5 times or more per day. In some examples, the compositions 1, 2, 3, 4, 5, 6 or 7 days per week.

In an exemplary method, the compound is administered to an eye tissue by injection, e.g., subconjunctival injection, subdermal injection around an eyelid, or periorbital injection. For example, the compound is administered at a dose of 1-300 μg per injection.

Also within the invention is a method of reducing the severity or treating an optical disease or disorder associated with reduced meibomian gland (MG) function in a subject by systemically administering to the subject a composition comprising a low oxygen mimetic compound, wherein reduced severity or treatment of said disease or disorder comprises increased tear secretion, increased tear volume, prolonged tear film break up time, improved tear osmolarity, improved gland expressibility, improved gland expressed oil quality and volume, increased thickness of tear film lipid layer, decreased ocular surface staining, decreased inflammation on the ocular surface, or alleviated ocular discomfort feeling of patients. For systemic administration, the compound is administered orally at a dose of 50-70 mg per oral administration or 1-10 mg/kg of body weight per injection. An exemplary oral dose does not exceed 3.0 mg/kg of body weight.

Using the methods described above, the local oxygen concentration at the MG in the subject is reduced to no more than 1%.

In addition to the members of the HIF-PHI class of compound, the methods utilize administration of dimethyloxalyglycine, desferrioxamine, cobalt (II) chloride (CoCl₂), of combinations thereof.

The invention also encompasses a method of promoting differentiation of a meibomian gland epithelial cell (MGEC) by i) reducing local oxygen concentration of the MGEC to no more than 1%; and/or ii) contacting the MGEC with an HIP-PHI. Such methods are carried out using the following exemplary compounds: Roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1), Dimethyloxalylglycine (DMOG), or combinations thereof.

The methods described herein are also useful for reducing weight or treating individuals characterized by obesity and/or high serum cholesterol. A method of treating obesity and/or reducing weight gain of a subject is carried out by administering to the subject a pharmaceutically effective dosage of an HIF-PHI agent or other compound that oxygen concentration. Such compounds may include Roxadustat (Roxa), dimethyloxalyglycine, desferrioxamine, cobalt (II) chloride (CoCl₂), of combinations thereof.

Dosage formulations are encompassed by the invention. For example, a dosage formulation for administration to a subject comprises Roxadustat (Roxa), e.g.,
i) in a therapeutically effective amount between about 0.1-100 mg/ml for administration as an eye drop for treatment of reduced meibomian gland (MG) function; or
ii) in a therapeutically effective amount between about 1-300 μg for administration as an injection for treatment of reduced meibomian gland (MG) function.

A dosage formulation for administration to a subject comprises Roxadustat (Roxa) in a therapeutically effective amount between about 50-500 mg for treatment of obesity and/or elevated serum cholesterol, wherein said effective amount is administered 1-5 times per week.

Various treatment systems and methods are disclosed for treating several dysfunctions. For example, a method of treating MGD is disclosed. The methods include reducing oxygen concentration in an eyelid environment of one or more dysfunctional MGs, as well as giving local or systemic drugs that lead to the generation of hypoxia-inducible factors (HIFs), e.g., HIF1α, in one or more dysfunctional MGs. These HIFs are induced by relative hypoxia and promote the function of MGs. The eyelid includes the skin and tarsal tissues between the eyebrow and the lower margin of the orbital cavity, and the MGs are located in the lower and upper eyelids. The blood supply for the eyelids are formed by anastomoses of the lateral palpebral arteries and medial palpebral arteries, branching off from the lacrimal artery and ophthalmic artery, respectively.

In one aspect, the instant disclosure provides a method of preventing or treating an optical disease or disorder related to a reduced meibomian gland (MG) function in a subject by administering to the subject a therapy. For example, such reduced meibomian gland (MG) function may be caused by meibomian gland dysfunction (MGD). In some embodiments, such therapy comprises reducing local oxygen concentration at the MG in the subject to no more than 5%, 3%, 2%, 1.5%, 1.3%, 1.2%, 1%, 0.5%, 0.3%, 0.2%, 0.1%, or less. In some embodiments, such therapy comprises reducing local oxygen concentration at the MG in the subject to no more than 1%. In some embodiments, such therapy comprises administering to the subject an agent capable of reducing oxygen concentration in the subject or mimicking the a low oxygen effect (e.g., low oxygen mimetic agents, which, without an intention to be limiting, may improve expression and/or stability of at least one of hypoxia-inducible factors, such as HIF1α). For example, such agent may comprise roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1), Dimethyloxalylglycine (DMOG), or combinations thereof. In some embodiments, the agent comprises a structure selected from the group consisting of Formula I, Formula II, Formula III, Formula IV, Formula V, Formula VI, Formula VII, and a pharmaceutically acceptable salt or prodrug thereof. A pharmaceutically effective amount of such agent may comprise about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of the agent, as described herein or found proper by a physician or a medical personnel. In some embodiments, such therapy comprises a combination of therapies described herein. For example, such therapy may comprise reducing local oxygen concentration at the MG in the subject to no more than 1% in combination with administering to the subject a pharmaceutically effective dosage of an agent capable of mimicking low oxygen concentration (e.g., by improving expression levels and/or stability of at least one HIFs, such as HIF1α). In some embodiments, such therapy comprises

i) reducing local oxygen concentration at the MG in the subject to no more than 1%;

ii) administering to the subject a dosage formulation comprising about 0.1 to about 100 mg/kg roxadustat (Roxa); or

iii) reducing local oxygen concentration at the MG in the subject to no more than 1% in combination with administering to the subject a pharmaceutically effective dosage of a low oxygen mimetic agent.

In some embodiments, the oxygen concentration is reduced to no more than 5%, 3%, 2%, 1.5%, 1.3%, 1.2%, 1%, 0.5%, 0.3%, 0.2%, 0.1%, or less. In some embodiments, the oxygen concentration is reduced to no more than 0.5% O₂ or 0.1% O₂.

In some embodiments, the low oxygen mimetic agent capable of mimicking low oxygen concentration comprises roxadustat (Roxa), dimethyloxalyglycine, desferrioxamine, cobalt (II) chloride (CoCl₂), or other agents described herein, or combinations thereof. In some embodiments, the agent comprises a structure selected from the group consisting of Formula I, Formula II, Formula III, Formula IV, Formula V, Formula VI, and Formula VII. Optionally, the agent comprises a pharmaceutically acceptable salt of Formula I, Formula II, c, Formula IV, Formula V, Formula VI, or Formula VII, or a prodrug of Formula I, Formula II, Formula III, Formula IV, Formula V, Formula VI, or Formula VII.

In some embodiments, the low oxygen mimetic agent comprises a dosage formulation comprising about 0.1 to about 100 mg/kg roxadustat (Roxa), or other compounds or molecules described herein. In some embodiments, the agent comprise a dosage formulation comprising about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of roxadustat (Roxa), or other compounds or molecules described herein.

In some embodiments, the dosage formulation is administrated locally or systematically to the subject. For example, the agent may be administered by topical, oral, subcutaneous, and/or intravenous (IV) routes.

In some embodiments, the therapy described herein increases levels of at least one of hypoxia-inducible factors and/or prevents degradation of at least one of hypoxia-inducible factors (HIFs). Such hypoxia-inducible factors may include, e.g., HIF-1α (HIF1A; hypoxia-inducible factor 1, alpha subunit), HIF-1α(ARNT; aryl hydrocarbon receptor nuclear translocator), HIF-2α (EPAS1; endothelial PAS domain protein 1), HIF-2α (ARNT2; aryl-hydrocarbon receptor nuclear translocator 2), HIF-3α (HIF3A; hypoxia inducible factor 3, alpha subunit), HIF-3α (ARNT3, aryl-hydrocarbon receptor nuclear translocator 3), etc. In some embodiments, the at least one of hypoxia-inducible factors comprises hypoxia-inducible factors 1α (HIF1α).

In some embodiments, the therapy described herein

i) promotes differentiation of a meibomian gland epithelial cell (MGEC);

ii) increases expression levels of sterol regulator element binding protein 1 (SREBP1);

iii) promotes an enlargement of lysosomes; and/or

iv) promotes deoxyribonuclease (DNase) II activity.

In some embodiments, the differentiation of a MGEC is measured by

i) intracellular lipid accumulation; and/or

ii) expression of Peroxisome proliferator-activated receptor gamma (PPAR-γ), Desmoglein and/or Desmocollin.

In some embodiments, the disease or disorder described herein comprises MG dysfunction (MGD), dry eye disease (DED), Sjogren's syndrome, systemic lupus erythematosus, or rheumatoid arthritis.

In another aspect, the instant disclosure provides a method of promoting differentiation of a meibomian gland epithelial cell (MGEC) by a method. Such method may comprise reducing local oxygen concentration of the MGEC to no more than 1%; and/or contacting the MGEC with a low oxygen mimetic agent.

In some embodiments, the oxygen concentration in the environment of the MGEC is reduced to no more than 5%, 3%, 2%, 1.5%, 1.3%, 1.2%, 1%, 0.5%, 0.3%, 0.2%, 0.1%, or less. In some embodiments, the oxygen concentration is reduced to no more than 0.5% O₂ or 0.1% O₂.

In some embodiments, the low oxygen mimetic agent comprises roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1), Dimethyloxalylglycine (DMOG), or combinations thereof. In some embodiments, the agent comprises roxadustat (Roxa), dimethyloxalyglycine, desferrioxamine, cobalt (II) chloride (CoCl₂), of combinations thereof. In some embodiments, the agent comprises a structure selected from the group consisting of Formula I, Formula II, Formula III, Formula IV, Formula V, Formula VI, and Formula VII. Optionally, the agent comprises a pharmaceutically acceptable salt of Formula I, Formula II, Formula III, Formula IV, Formula V, Formula VI, or Formula VII, or a prodrug of Formula I, Formula II, Formula III, Formula IV, Formula V, Formula IV or Formula VII.

In some embodiments, the low oxygen mimetic agent comprises a dosage formulation comprising about 0.1 to about 100 mg/kg roxadustat (Roxa), or other compounds or molecules described herein. In some embodiments, the agent comprise a dosage formulation comprising about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of roxadustat (Roxa), or other compounds or molecules described herein.

In some embodiments, the method described herein increases levels of at least one of hypoxia-inducible factors (HIFs), such as hypoxia-inducible factor 1α (HIF1α) and/or prevents degradation of at least one of HIFs, such as HIF1α in the MGEC.

In some embodiments, the method described herein

i) promotes differentiation of the MGEC;

ii) increases expression levels of sterol regulator element binding protein 1 (SREBP1) in the MGEC;

iii) promotes an enlargement of lysosomes in the MGEC; and/or

iv) promotes deoxyribonuclease (DNase) II activity in the MGEC.

In some embodiments, the differentiation of the MGEC is measured by

i) intracellular lipid accumulation; and/or

ii) expression of Peroxisome proliferator-activated receptor gamma (PPAR-γ), Desmoglein and/or Desmocollin in the MGEC.

In another aspect, the instant disclosure provides a method of treating obesity and/or reducing weight gain of a subject by a method. Such method may comprise reducing local oxygen concentration in the subject, e.g., near the eyes or eyelids of the subject, to no more than 1%, administering to the subject an agent capable of mimicking oxygen concentration, or a combination thereof. In some embodiments, the method comprises administering to the subject a pharmaceutically effective dosage of a low oxygen mimetic agent capable of mimicking low oxygen concentration in the MG.

In some embodiments, the local oxygen concentration in the subject, e.g., near the eyes or eyelids of the subject, is reduced to no more than 5%, 3%, 2%, 1.5%, 1.3%, 1.2%, 1%, 0.5%, 0.3%, 0.2%, 0.1%, or less. In some embodiments, such oxygen concentration is reduced to no more than 0.5% O₂ or 0.1% O₂.

In some embodiments, the low oxygen mimetic agent comprises roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1), Dimethyloxalylglycine (DMOG), or combinations thereof. In some embodiments, the agent comprises roxadustat (Roxa), dimethyloxalyglycine, desferrioxamine, cobalt (II) chloride (CoCl₂), of combinations thereof. In some embodiments, the agent comprises a structure selected from the group consisting of Formula I, Formula II, Formula III, Formula IV, Formula V, Formula VI, and Formula VII. Optionally, the agent comprises a pharmaceutically acceptable salt of Formula I, Formula II, Formula III, Formula IV, Formula V, Formula VI, or Formula VII, or a prodrug of Formula I, Formula II, Formula III, Formula IV, Formula V, Formula VI, or Formula VII.

In some embodiments, such agent comprises a dosage formulation comprising about 0.1 to about 100 mg/kg roxadustat (Roxa), or other compounds or molecules described herein. In some embodiments, the agent comprise a dosage formulation comprising about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of roxadustat (Roxa), or other compounds or molecules described herein.

In some embodiments, the dosage formulation describe herein is administrated locally or systematically to the subject. For example, the agent may be administered by oral, subcutaneous, and/or intravenous (IV) routes.

In some embodiments, the method described herein increases levels of at least one of hypoxia-inducible factors (HIFs), such as hypoxia-inducible factor 1α (HIF1α) and/or prevents degradation of at least one of HIFs, such as HIF1α, in the subject, e.g., near the eyes or eyelids of the subject.

In some embodiments, the method described herein increases levels of neutral lipid droplets and/or nonpolar lipids in the meibomian gland (MG) of the subject, promotes differentiation of the meibomian gland epithelial cells (MGECs) in the subject; increases tear volume of the subject; and/or increases the size of the meibomian gland (MG) of the subject.

In some embodiments, the method described herein

i) promotes differentiation of an MGEC;

ii) increases expression levels of sterol regulator element binding protein 1 (SREBP1);

iii) promotes an enlargement of lysosomes; and/or

iv) promotes deoxyribonuclease (DNase) II activity

in the subject, e.g., near the eyes or eyelids of the subject.

In some embodiments, the differentiation of the MGEC in the subject, e.g., near the eyes or eyelids of the subject, is measured by

i) intracellular lipid accumulation; and/or

ii) expression of Peroxisome proliferator-activated receptor gamma (PPAR-γ), Desmoglein and/or Desmocollin.

In some embodiments, the hypoxia-mimetic agent described herein promotes expression and/or stability of at least one HIFs (such as HIF1α) in the subject. For example, the agent may increase the expression levels and/or stability of at least one HIFs (such as HIF1α) to about 10%, 15%, 20%, 30%, 40%, 50%, 60%, 70%, 80%, 90%, 100%, 150%, 200%, 300%, 400%, 500%, or more than normal or control levels or stability, e.g., levels or stability in a subject with the same disease or disorder but not treated with the agent.

In some embodiments, the agent described herein prevents the percentage of weight gain and/or promote weight loss in the subject. For example, the agent may prevents the percentage of weight gain and/or promote weight loss in the subject to about 90%, 85%, 80%, 75%, 70%, 65%, 60%, 55%, 50%, 45%, 40%, 35%, 30%, 25%, 20%, 15%, 10%, or less, of normal or control levels, e.g., levels in a healthy subject or a subject with the same disease or disorder but not treated with the agent.

In another aspect, the instant disclosure provides a dosage formulation for administration to a subject, wherein the formulation comprises roxadustat (Roxa), or other compounds or molecules described herein, in a therapeutically effective amount between about 0.1 mg/kg about 100 mg/kg. In some embodiments, the subject has an optical disease or disorder related to a reduced meibomian gland (MG) function; and/or obesity, or other disease or disorder related to high serum cholesterol and/or abnormal weight gain (e.g., overweight).

In some embodiments, the formulation comprises roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1), Dimethyloxalylglycine (DMOG), or combinations thereof. In some embodiments, the agent comprises roxadustat (Roxa), dimethyloxalyglycine, desferrioxamine, cobalt (II) chloride (CoCl₂), of combinations thereof. In some embodiments, the formulation comprises a compound comprising a structure selected from the group consisting of Formula I, Formula II, Formula III, Formula IV, Formula V, Formula VI, and Formula VII. Optionally, the agent comprises a pharmaceutically acceptable salt of Formula I, Formula II, Formula III, Formula IV, Formula V, Formula VI, or Formula VII, or a prodrug of Formula I, Formula II, Formula III, Formula IV, Formula V, Formula VI, or Formula VII.

In some embodiments, the formulation comprises about 0.1 to about 100 mg/kg roxadustat (Roxa), or other compounds or molecules described herein. In some embodiments, the formulation comprise about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of roxadustat (Roxa), or other compounds or molecules described herein.

In some embodiments, the dosage formulation describe herein is administrated locally or systematically to the subject. For example, the agent may be administered by topical, oral, subcutaneous, and/or intravenous (IV) routes.

In some embodiments, the dosage formulation increases levels of at least one of hypoxia-inducible factors (HIFs), such as hypoxia-inducible factor 1α (HIF1α), and/or prevents degradation of at least one of HIFs, such as HIF1α, in the subject, e.g., near the eyes or eyelids of the subject.

In some embodiments, the dosage formulation

i) promotes differentiation of an MGEC;

ii) increases expression levels of sterol regulator element binding protein 1 (SREBP1);

iii) promotes an enlargement of lysosomes; and/or

iv) promotes deoxyribonuclease (DNase) II activity

in the subject, e.g., near the eyes or eyelids of the subject.

In some embodiments, the differentiation of the MGEC in the subject, e.g., near the eyes or eyelids of the subject, is measured by

i) intracellular lipid accumulation; and/or

ii) expression of Peroxisome proliferator-activated receptor gamma (PPAR-γ), Desmoglein and/or Desmocollin.

In some embodiments, the dosage formulation reduces serum cholesterol in the subject. For example, the agent may reduce serum cholesterol in the subject to about 90%, 85%, 80%, 75%, 70%, 65%, 60%, 55%, 50%, 45%, 40%, 35%, 30%, 25%, 20%, 15%, 10%, or less, of normal or control levels, e.g., levels in a healthy subject or a subject with the same disease or disorder but not treated with the agent.

In some embodiments, the dosage formulation described herein prevents the percentage of weight gain and/or promote weight loss in the subject. For example, the agent may prevents the percentage of weight gain and/or promote weight loss in the subject to about 90%, 85%, 80%, 75%, 70%, 65%, 60%, 55%, 50%, 45%, 40%, 35%, 30%, 25%, 20%, 15%, 10%, or less, of normal or control levels, e.g., levels in a healthy subject or a subject with the same disease or disorder but not treated with the agent.

In some embodiments, the subject is further treated with a therapy to reduce local oxygen concentration at the MG in the subject to a hypoxia level, e.g., no more than 1% O₂.

In some embodiments, the dosage formulation is for local or systemic administration. For example, the dosage formulation is for topical, oral, subcutaneous, or intravenous (IV) administration.

In some embodiments, the dosage formulation described herein increases levels of neutral lipid droplets and/or nonpolar lipids in the meibomian gland (MG) of the subject. In some embodiments, the nonpolar lipids comprise triglyceride and/or free fatty acids. In some embodiments, the dosage formulation described herein promotes differentiation of the meibomian gland epithelial cells (MGECs) in the subject. In some embodiments, the promoted differentiation of MGECs is measured by the decrease of cell number of MGECs. In some embodiments, the dosage formulation described herein increases tear volume of the subject. In some embodiments, the dosage formulation described herein increases the size of the meibomian gland (MG) of the subject. In some embodiments, the function(s) of the dosage formulation described herein is to increase at least one feature, as described herein, to about 10%, 15%, 20%, 25%, 30%, 35%, 40%, 45%, 50%, 55%, 60%, 65%, 70%, 75%, 80%, 85%, 90%, 95%, 100%, 150%, 200%, or more, than the normal or control levels without the treatment of the dosage formulation. In some embodiments, the function(s) of the dosage formulation described herein is to reduce at least one feature, as described herein, to about 90%, 85%, 80%, 75%, 70%, 65%, 60%, 55%, 50%, 45%, 40%, 35%, 30%, 25%, 20%, 15%, 10%, or less, than the normal or control levels without the treatment of the dosage formulation.

In some embodiments, the subject is a human, non-human primate, mouse, rat, dog, cat, horse, cattle, sheep, pig, chicken, or goat.

In another aspect, the instant disclosure provides a kit comprising a dosage formulation for administration to a subject, wherein the formulation comprises roxadustat (Roxa), or other compounds or molecules described herein, in a therapeutically effective amount between about 0.1 mg/kg about 100 mg/kg.

As used herein, “kits” are understood to contain at least one non-standard laboratory reagent, such as the agent described herein, for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The composition and/or the dosage formulation described herein is in the form of a tablet, a capsule, a powder, a beverage, or an infant formula.

By “agent” is meant any small molecule chemical compound, antibody, nucleic acid molecule, or polypeptide, or fragments thereof. In some embodiments, the agent comprises a structure selected from the group consisting of Formula I, Formula II, Formula III, Formula IV, Formula V, Formula VI, and Formula VII. Optionally, the agent comprises a pharmaceutically acceptable salt of Formula I, Formula II, Formula III, Formula IV, Formula V, Formula VI, or Formula VII, or a prodrug of Formula I, Formula II, Formula III, Formula IV, Formula V, Formula VI, or Formula VII.

There are different definitions for hypoxia. The terms physiological, modest, moderate and severe hypoxia and anoxia have been used to designate 10-14, 2.5, 0.5, 0.1 and 0% O₂, respectively [Evans et al. (2006) The Journal of Investigative Dermatology 126:2596-2606]. In this disclosure, the terms “hypoxia” or “hypoxic environment” refer to an O₂ concentration of less than 5%. This usage is consistent with that of other studies [McKeown (2014) Br J Radiol 87:20130676].

Exemplary 1% O₂ concentration was used for the experiments disclosed in this application. This contrasts with the 3% O₂ used in previous study of relative hypoxia and the MG [Liu et al. (2019) The ocular surface 17(2):310-317]. The rationale for this change was that it was discovered that the mouse MG stains positively for pimonidazole, a biomarker for hypoxia in vivo [Samoszuk et al. (2004) The Journal of Histochemistry and Cytochemistry: Official Journal of the Histochemistry Society 52:837-839; Olive et al. (2001) Acta Oncologica 40:917-923], and stains tissues that contain O₂ levels below 1.3% [Gross et al. (1995) International Journal of Cancer Journal international du cancer 61:567-573]. Consequently, 1% O₂ was chosen to better imitate the O₂ conditions in the MG in vivo.

As used herein, an “isolated” or “purified” nucleic acid molecule, polynucleotide, polypeptide, or protein, small molecules, compounds, or other agents described herein is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated protein or polypeptide is free of the amino acid sequences that flank it in its naturally occurring state. Purified also defines a degree of sterility that is safe for administration to a human subject, e.g., lacking infectious or toxic agents.

Similarly, by “substantially pure” is meant a nucleotide, polypeptide, small molecule, compound, or other agents described herein, that has been separated from the components that naturally accompany it or accompany it during chemical productions or processing. Typically, the nucleotides, polypeptides, small molecules, compounds, or other agents described herein, are substantially pure when they are at least 60%, 70%, 80%, 90%, 95%, or even 99%, by weight, free from the proteins, naturally occurring organic molecules, chemical precursors, or other chemicals.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

The terms “subject,” “patient,” “individual,” and the like as used herein are not intended to be limiting and can be generally interchanged. That is, an individual described as a “patient” does not necessarily have a given disease, but may be merely seeking medical advice.

The term “reduce,” “attenuate,” “promote,” or “increase” is meant to alter negatively or positively, respectively, by at least 5%. An alteration may be by 5%, 10%, 25%, 30%, 50%, 75%, 90%, 100%, or even more (for positive alternations).

As used herein, a “symptom” associated with a disorder includes any clinical or laboratory manifestation associated with the disorder, and is not limited to what the subject can feel or observe.

By the terms “effective amount” and “therapeutically effective amount” of a formulation or formulation component is meant a sufficient amount of the formulation or component to provide the desired effect. For example, by “an effective amount” is meant an amount of an agent described herein (such as Roxa) to prevent, attenuate, and/or inhibit, a cancer (e.g., preventing, attenuating, and/or inhibiting the proliferation of a cancer cell) in a subject. Ultimately, the attending physician or veterinarian decides the appropriate amount and dosage regimen.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the singular forms “a,” “an,” and “the” include the plural reference unless the context clearly dictates otherwise. Thus, for example, a reference to “a disease,” “a disease state”, or “a compound” is a reference to one or more such embodiments, and includes equivalents thereof known to those skilled in the art and so forth.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, Genbank/NCBI accession numbers, and other references mentioned herein are incorporated by reference in their entirety. In the case of conflict, the present specification, including definitions, will control. In addition, the materials, methods, and examples are illustrative only and not intended to be limiting.

### BRIEF DESCRIPTION OF THE DRAWINGS

FIG. 1 is a group of microscopic images illustrating the identification of HIF1α protein in human MG acini. Human eyelid tissues were collected and processed. Frozen sections of human MGs were incubated with an antibody specific for HIF1α and then replaced with mouse IgG for negative controls, prior to 4′,6-diamidino-2-phenylindole blue nuclear staining for microscopy. FIG. 1 shows HIF1α and 4′,6-diamidino-2-phenylindole co-staining within acinar epithelial cell nuclei (arrow heads in the “Merge” panel). Photographs from one experiment are shown as a representative of three independent studies.

FIGS. 2A and 2B are Western Blot images and bar charts illustrating effects of 1% O₂ and Roxa on HIF1α expression in IHMGECs. In FIG. 2A, IHMGECs lysates after different treatment (“CTL” for control) were prepared for detecting the HIF1α expression by an anti-HIF1α antibody using western blot. The lysate of HepG2 cells treated with CoCl₂ for 4 hours was used as a positive control for the HIF1α antibody. FIG. 2B shows a bar chart comparing the HIF1α expression levels, detected as band intensity and normalized to β-actin band intensity, in FIG. 2A. (*P<0.05, **P<0.001, ***P<0.0001; n=3 experiments) Both low O₂ (1%) and Roxa significantly increased the HIF1α expression in IHMGECs after 6 hours of treatment. The combination of low O₂ and Roxa induced a greater effect.

FIGS. 3A-3C are microscopic images and bar charts illustrating influence of 1% O₂ and Roxa on the accumulation of neutral lipid-containing droplets in IHMGECs. IHMGECs were exposed to normoxic conditions (21% O₂), low O₂ (1%), or Roxa, alone or in combination, for 4 days and then stained with 0.3% ORO for lipid droplets. FIG. 3A shows the microscopy view (20×) for round and lucent vesicles in cells exposed to 1% O₂, Roxa, or their combination. FIG. 3B shows the microscopy view (40×) for vesicles that stained positive for ORO staining. FIG. 3C shows a bar chart comparing the area of ORO staining in IHMGECs. One percent O₂, Roxa, and their combination all significantly increased the area of ORO staining in IHMGECs (*P<0.0001; n=3 experiments). There were no significant differences between these treatment groups. Arbitrary units stand for size of the stained areas. Scale bar=50 μM.

FIGS. 4A-4C are bar charts and images illustrating the effect of low O₂ and Roxa on the expression of neutral lipids and phospholipids in IHMGECs. IHMGECs were exposed to 21% O₂, 1% O₂, or Roxa, alone or in combination, for 4 days and then the lipid extracts were analyzed by HPTLC. Band intensity was measured with ImageJ; control band intensity was set to 1, and data (mean±SE) are reported as fold-change compared to controls. The data were pooled from three independent experiments (*P<0.05, **P<0.01). The triglyceride content (FIG. 4A) or free fatty acid content (FIG. 4B) in IHMGECs were compared in bar charts. Roxa significantly increased triglyceride and free fatty acid contents in IHMGECs in both 21% O₂ and 1% O₂ conditions. Arbitrary units stand for the normalized intensity of different bands to that of control. FIG. 4C shows that one percent O₂ and Roxa appeared to increase the expression of unidentified bands in nonpolar (arrow in the upper panel) and polar (arrow head in the lower panel) lipids, respectively.

FIGS. 5A-5C are bar charts illustrating the impact of low O₂ and Roxa on DNase II activity and cell number in IHMGECs. IHMGECs were treated with 21% O₂, 1% O₂, or Roxa, alone or in combination, for 10 days, and the DNase II activity in cell lysates and culture media was then analyzed by single radial enzyme diffusion assay. DNase II activity was significantly increased by 1% O₂, Roxa, and their combination in both cell lysate (FIG. 5A) and supernatant (FIG. 5B). 1% O₂ showed a significantly higher increase in DNase II activity compared with Roxa, and their combination treatment had an amplified effect. FIG. 5C shows that 1% O₂, Roxa, and combination also significantly decreased the cell number after 10 days (*P<0.01, **P<0.001, ***P<0.0001; n=3 experiments). The y-axis represents the normalized size of the dark circle under an UV light to cell numbers.

FIG. 6 is a bar chart illustrating that Roxa treatment significantly increased the tear volume in the mice comparing to the control group and the WT mice (*p<0.01). Two groups of ApoE KO mice (vehicle-treated ApoE −/−, as control, and Roxa-treated ApoE −/−), as well as wild-type C57BL/6J mice, were measured for weight, tear volume, corneal fluorescein staining and tear breakup time (TBUT). ApoE −/− mice were randomly divided into 2 groups (n=8 mice/group): control and Roxa-treated. The ApoE−/− mice were treated with vehicle control (2% DMSO in sterile saline, subcutaneous injection) or Roxa (10 mg/kg, subcutaneous injection). The injections were administrated three times/week; on Monday, Wednesday, and Friday mornings; for 12 weeks. At the end of the study, the weight, tear volume, corneal fluorescein staining and tear breakup time (TBUT) were weighted again for comparison. The bar chart in FIG. 6 compares mean tear volume of each group of mice after treatment.

FIG. 7 is a bar chart illustrating that Roxa treatment significantly reduced the meibomian gland loss in the mice eyelids comparing to the control group (* p<0.05). After the Roxa treatment, as described in FIG. 6, the eyelids of each mouse were dissected to assess meibomian gland size and morphology. The MG areas were compared between the Roxa-treated mice (“Roxa”) and the vehicle-treated mice (“CTL”), shown in bar graphs.

FIGS. 8A-8B are bar charts illustrating that Roxa treatment significantly reduced the percentage of the serum cholesterol level (FIG. 8A) and the percentage of weight gain (FIG. 8B) in the mice as used for the above studies (*p<0.05). The whole blood samples of mice were collected by cardiac puncture after sacrificing. The blood samples were allowed to clot by leaving it undisturbed at room temperature for 15 minutes. The clot was removed by centrifuging at 1,000-2,000×g for 10 minutes in a refrigerated centrifuge. The resulting supernatant was designated serum. Then the cholesterol level in the serum was evaluated by using a commercially available cholesterol fluorometric assay kit (#10007640, Cayman Chemical, Mich.). The weight of each mice was measured before and after the experiment. The percentage of weight gain was compared between the Roxa-treated mice and the vehicle-treated mice.

FIG. 9 is the Ocular Surface Disease Index (OSDI) 12-item questionnaire.

FIG. 10 is the Oxford Schema for grading corneal and conjunctival staining.

FIG. 11 is a questionnaire given to subjects to diagnose Meibomian Gland Dysfunction.

FIG. 12 is a flow diagram showing the sequence of clinical tests performed to evaluate Meibomian Gland function.

### DETAILED DESCRIPTION

Low O₂ is usually considered to have adverse effects on human health. However, under certain conditions, low O₂ may be actually beneficial for diseased tissues, according to the latest discoveries. In fact, low O₂ therapy (also called as hypoxia therapy) has been used in both animal models and phase III clinical trials to treat various conditions in recent years, such as mitochondrial disease, wound healing, retinopathy of prematurity, corneal endothelial cell protection, anemia and tissue regeneration. Low O₂ therapy includes reducing the O₂ concentration, either systemically or locally in a tissue, or inducing a cellular response by using hypoxia mimetic drugs.

The instant disclosure includes, at least, a method for using low O₂ therapy as a new treatment for meibomian gland dysfunction (MGD) and other related diseases or disorders. In fact, MGD is the major cause of dry eye disease (DED), which afflicts countless people throughout the world (i.e., greater than 40 million in the USA), and is one of the most frequent causes of patient visits to eye care practitioners. Prevalence of this disease ranges from approximately 5% up to 75% in different populations, with more affected women than men. Prevalence is also higher in Asian and aging populations, though younger people are increasingly exhibiting symptoms. As the world population is expected to increase from 7.2 billion in 2012 to between 8.3-10.9 billion by 2050, the incidence of DED is expected to rise. According to The Lancet Series on Aging, published in 2014, 2 billion people will be aged 60 years or older by the year 2050. Assuming a prevalence of 25%, 500 million people will have dry eye disease in this demographic alone. The current burden of DED for the USA healthcare system is estimated to be over $3.8 billion, and, because of diminished productivity, $55.4 billion for the USA overall. It has been estimated that manufacturers' global revenues in the dry eye treatments market will climb from $4.6 billion in 2018 to $6.2 billion in 2023. The DED market has been divided into three broad segments: procedures, over-the-counter (OTC) lubricants, and prescription pharmaceuticals. Revenues from OTC lubricants and prescription pharmaceuticals are forecast to grow moderately—in the range of 4.3 percent to 4.5 percent a year. A much higher cumulative annual growth rate of 21.4 percent is expected in procedure revenues.

Patients with DED continuously experience dryness, stinging, burning, chronic pain, and blurred vision. Moderate to severe DED is associated with significant pain, role limitations, low vitality and poor general health. These symptoms significantly impair quality of life. Additionally, patients with DED frequently report significant disturbances in their psychiatric state, showing symptoms of anxiety and depression. A previous diagnosis of DED, or frequent experiences of dry eye symptoms, has been associated with depression and suicidal ideation. There is currently no global cure for MGD or DED.

Treatments for MGD include the LipiFlow thermal pulsation system, warm compression, lid cleansing, and the off-label use of antibiotics or intense pulsed light therapy. In addition to these MGD-specific treatments, patients are often prescribed one of the three FDA-approved drugs for DED. All of these are immunosuppressants, which target inflammatory pathways and are designed to alleviate the signs and symptoms of DED. Two of these drugs share the same major ingredient, but with differences in dose and formation. However, the pathophysiology of MGD and DED, and the mechanisms of current treatments are still unclear, and the long-term effects of these treatments are unclear. Although MGD can induce inflammation at the ocular surface, the role of inflammation in the etiology of MGD is controversial and uncertain. In addition, the vast majority of interventional clinical trials of anti-inflammatory and immunomodulatory agents have failed to meet their primary endpoints. It appears that inflammation is not the ideal target for MGD and DED treatment.

Compared to current treatments for MGD, the instant disclosure provides, at least, a method to target a completely different pathway. It was recently found for the first time that healthy human MGs exist in a physiologically hypoxic environment, and that a low O₂ environment significantly promotes the function of human MG epithelial cells (HMGECs). Low oxygen mimetic drugs can duplicate the function-promoting effect of a low O₂ environment in HMGECs. This discovery reveals a new aspect of the biology of the MG and pathophysiology of MGD, and also exposes the potential for developing new treatments. The instant disclosure provides, at least, a method to use low O₂ therapy to promote the function of diseased MGs by correcting the disease upstream, suggesting that by inducing the physiologically hypoxic environment for MG tissue, normal MG function may be restored.

The instant disclosure provides, at least, a method to treat patients with MGD and DED with a new therapeutic modality—a low O₂ therapy. Low O₂ therapy includes reducing the O₂ concentration, either systemically or locally in a tissue, or inducing a cellular response by using hypoxia mimetic drugs, such as Roxadustat (Roxa). Even though it may seem counterintuitive, the instant disclosure provides that low O₂ promotes the function of MGs, and Roxa can duplicate the effect of low O₂ on MGs. In order to translate the treatment into the clinical setting, Roxa may be applied locally around the MGs. Because the MG is a superficial tissue located in the eyelids, it is not difficult to apply Roxa topically and induce a local low O₂ reaction in the MGs. In addition, because MGD and DED do not directly impact other organs, Roxa, or other molecules disclosed herein, can be localized at the ocular surface to avoid systemic effects. The effectiveness of systemic use of Roxa, or other molecules disclosed herein, may be tested in treating MGD, and if successful, Roxa, or other molecules disclosed herein, may be further developed into an eye drop or eye ointment. If the project is successful, patients could buy the drug with prescription and use it at home.

It was discovered, for the first time, that human MGs exist in a physiologically hypoxic environment, and low O₂ environment significantly promotes the function of HMGECs. This discovery significantly advances the current understanding of the pathophysiology of MGD. Several low oxygen mimetic drugs, such as Roxa, can duplicate the positive effect of low O₂ environment in HMGEC function. Roxa showed a stronger lipid-inducing effect in HMGECs than other low oxygen mimetic drugs. Roxa is now marketed for the treatment of anemia with chronic kidney disease in China. Thus, Roxa has passed the toxicity tests for both clinical trial and animal studies.

### Agents

This specification describes at least one agent that may be administered to the subject in a pharmaceutically effective amount, a pharmaceutical composition, and/or a dosage formulation. In vitro, such agent may mimic a low oxygen (hypoxia) concentration in the environment of a meibomian gland epithelial cell (MGEC) (i.e., as low oxygen mimetic agents, such as roxadustat/Roxa), promote the differentiation of the MGEC (thus decreasing the MGEC number), increases levels of at least one of hypoxia-inducible factors (HIFs), such as HIF1α and/or prevents degradation of at least one of hypoxia-inducible factors (HIFs), such as HIF1α, in the MGEC, increase the size of the MG, increase the levels of neutral lipid droplets and/or nonpolar lipid acids in the MGEC, or combinations thereof. The agent may have the similar in vivo functions in a subject. For example, such agent may increase the MG function in a subject having reduced MG functions and/or a disease or disorder related to reduced MG functions, increase tear volume, increase the size of the MG, decrease the serum cholesterol level, decrease the percentage of weight gain, or combinations thereof. In general, the agents described herein is capable of mimicking low oxygen concentration at the MG in a subject, increasing MG functions in a subject, decreasing serum cholesterol levels, and/or decreasing the percentage of weight gain in the subject.

The agent described herein may comprise, for example, roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1, Dimethyloxalylglycine (DMOG), or combinations thereof. In some embodiments, the agent comprises an HIF prolyl hydroxylases inhibitor (HIF-PHI). In some embodiments, the HIF-PIH comprises roxadustat (Roxa), daprodustat/GSK1278863, molidustat/BAY 85-3934, vadadustat/AKB-6548, desidustat/ZYAN1.

A pharmaceutically effective amount of such agent may comprise about 0.1 to about 100 mg/kg, about 0.1 to about 50 mg/kg, about 0.1 to about 20 mg/kg, about 0.1 to about 10 mg/kg, about 0.1 to about 5 mg/kg, about 0.5 to about 100 mg/kg, about 0.5 to about 50 mg/kg, about 0.5 to about 20 mg/kg, about 0.5 to about 10 mg/kg, about 0.5 to about 5 mg/kg, about 1 to about 100 mg/kg, about 1 to about 50 mg/kg, about 1 to about 20 mg/kg, about 1 to about 10 mg/kg, about 1 to about 5 mg/kg, about 5 to about 10 mg/kg, about 5 to about 20 mg/kg, about 5 to about 50 mg/kg, about 5 to about 100 mg/kg, or other amount of the agent.

In some embodiments, the agents described herein comprises roxadustat (Roxa, a.k.a., FG-4592, ASP1517, AZD9941). Roxa has a structure as Formula I below:

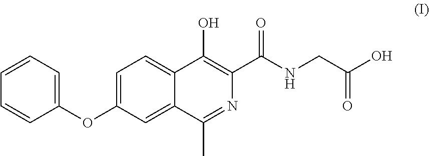 [SMILES: Cc1nc(C(=O)NCC(=O)O)c(O)c2ccc(Oc3ccccc3)cc12]

In some embodiments, the agents described herein comprises roxadustat (Roxa) comprising a structure of Formula I, or a pharmaceutically acceptable salt or prodrug thereof.

In some embodiments, the agent may have a structure as Formula II below.

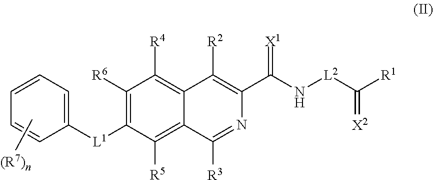

In Formula II:

each X¹ and X² is independently O or S;

each L¹ and L² is —O—, —NR^{a}—, or R^{c}-substituted or unsubstituted alkylene (e.g., C₁-C₈ alkyl, C₁-C₆ alkylene, or C₁-C₄ alkylene);

R¹ is —OR^{a}, NR^{a}R^{b}, R^{c}-substituted or unsubstituted alkyl (e.g., C₁-C₈ alkyl, C₁-C₆ alkyl, or C₁-C₄ alkyl), or R^{c}-substituted or unsubstituted heteroalkyl (e.g., 2 to 8 membered heteroalkyl, 2 to 6 membered heteroalkyl, or 2 to 4 membered heteroalkyl);

each R², R³, R⁴, R⁵, R⁶, and R⁷ is independently hydrogen, halogen, —OR^{a}, NR^{a}R^{b}, R^{c}-substituted or unsubstituted alkyl (e.g., C₁-C₈ alkyl, C₁-C₆ alkyl, or C₁-C₄ alkyl), or R^{c}-substituted or unsubstituted heteroalkyl (e.g., 2 to 8 membered heteroalkyl, 2 to 6 membered heteroalkyl, or 2 to 4 membered heteroalkyl);

each R^{a} and R^{b} is independently hydrogen, or unsubstituted alkyl (e.g., C₁-C₈ alkyl, C₁-C₆ alkyl, or C₁-C₄ alkyl);

R^{c} is halogen, oxo, —N₃, —CN, —N₃, —OH, —NH₂, —COOH, —CONH₂, —NO₂, —SH, —SO₂H, —SO₃H, or unsubstituted alkyl; and n is an integer of 0 to 5.

In some embodiments, X¹ is O. In some embodiments, X² is O. In some embodiments, X¹ is S. In some embodiments, X² is S.

In some embodiments, L¹ is —O—, —NH—, or unsubstituted C₁-C₄ alkylene. In some embodiments, L² is —O—, —NH—, or unsubstituted C₁-C₄ alkylene. In some embodiments, L¹ is —O—. In some embodiments, L² is methylene or ethylene.

In some embodiments, le is —OH, —OCH₃, —NH₂, —NHCH₃, NR^{a}R^{b}, or unsubstituted C₁-C₄ alkyl. In some embodiments, R¹ is —OH or —OCH₃.

In some embodiments, R² is hydrogen, —OR^{a}, —NR^{a}R^{b}, or unsubstituted C₁-C₄ alkyl. In some embodiments, R³ is hydrogen, —OR^{a}, —NR^{a}R^{b}, or unsubstituted C₁-C₄ alkyl. In some embodiments, each R^{a} and R^{b} is independently hydrogen or unsubstituted C₁-C₄ alkyl. In some embodiments, R² is —OH, —OCH₃, methyl, or ethyl. In some embodiments, R³ is —OH, —OCH₃, methyl, or ethyl. In some embodiments, R² is —OH. In some embodiments, R³ is methyl.

In embodiments, each R⁴, R⁵, R⁶, and R⁷ is independently hydrogen, —OH, or unsubstituted C₁-C₄ alkyl. In embodiments, each R⁴, R⁵, R⁶, and R⁷ are hydrogen.

In some embodiments, the agent may have a structure as Formula III below.

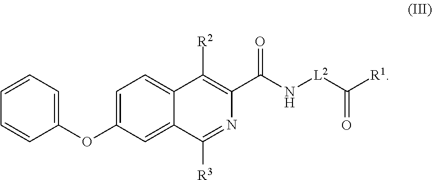

R¹, R², R³, and L² are as described herein.

In some embodiments, R¹ is —OH. In some embodiments, L² is unsubstituted methylene or ethylene. In some embodiments, each of R² and R³ is independently —OH or methyl. In some embodiments, R² is —OH and R³ is methyl.

In some embodiments, the agent described herein comprises a structure selected from the group consisting of Formula I, Formula II, Formula III, and a pharmaceutically acceptable salt or prodrug thereof.

In some embodiments, the agents described herein comprises daprodustat (a.k.a., GSK1278863). Daprodustat has a structure as Formula IV below:

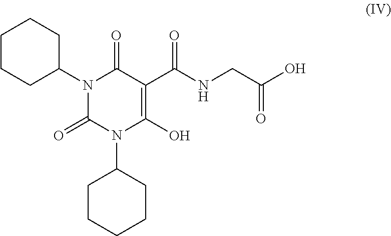 [SMILES: O=C(O)CNC(=O)c1c(O)n(C2CCCCC2)c(=O)n(C2CCCCC2)c1=O]

In some embodiments, the agents described herein comprises daprodustat/GSK1278863 comprising a structure of Formula IV, or a pharmaceutically acceptable salt or prodrug thereof.

In some embodiments, the agents described herein comprises molidustat (a.k.a., Bay 85-3934). Molidustat has a structure as Formula V below:

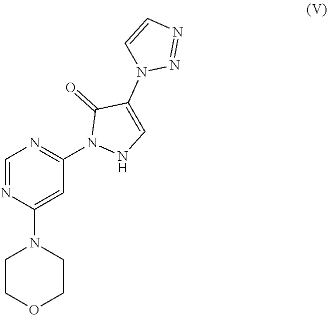 [SMILES: O=c1c(-n2ccnn2)c[nH]n1-c1cc(N2CCOCC2)ncn1]

In some embodiments, the agents described herein comprises molidustat/Bay 85-3934 comprising a structure of Formula V, or a pharmaceutically acceptable salt or prodrug thereof.

In some embodiments, the agents described herein comprises vadadustat (a.k.a., AKB-6548, PG-1016548). Vadadustat has a structure as Formula VI below:

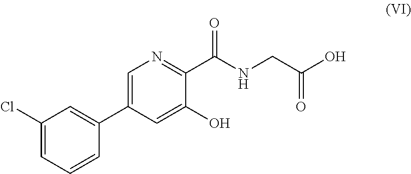 [SMILES: O=C(O)CNC(=O)c1ncc(-c2cccc(Cl)c2)cc1O]

In some embodiments, the agents described herein comprises vadadustat comprising a structure of Formula VI, or a pharmaceutically acceptable salt or prodrug thereof.

In some embodiments, the agents described herein comprises desidustat (a.k.a., ZYAN1). Desidustat has a structure as Formula VII below:

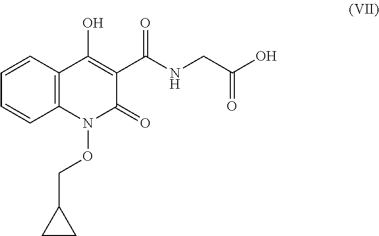 [SMILES: O=C(O)CNC(=O)c1c(O)c2ccccc2n(OCC2CC2)c1=O]

In some embodiments, the agents described herein comprises desidustat comprising a structure of Formula VII, or a pharmaceutically acceptable salt or prodrug thereof.

### Low Oxygen Concentration

The instant disclosure provides a method to increase MG function and/or treat a disease or disorder related to reduced MG function in a subject by application of low oxygen environment to the MG or the subject. There are different definitions for hypoxia; the terms physiological, modest, moderate and severe hypoxia and anoxia have been used to designate 10-14, 2.5, 0.5, 0.1 and 0% O₂, respectively [Evans et al. (2006) The Journal of Investigative Dermatology 126:2596-2606]. In the instant disclosure, the terms “hypoxia” or “hypoxic environment” refer to an O₂ concentration of less than 5%, unless expressly taught otherwise. This usage is consistent with that of other studies [McKeown (2014) Br J Radiol 87:20130676]. In some embodiments, an oxygen concentration lower than the physiological oxygen concentration in the mice MG (i.e., 1.3%) is used, such as less than 1.3%, 1%, 0.5%, 0.1%, or less oxygen. A low O₂ environment (generally under 5% O₂) triggers a hypoxic response pathway in mammalian cells, which is centered on the regulated expression of hypoxia-inducible factor (HIF). HIF, which consists of α and β subunits, is the central player that regulates hypoxic responses. To date, three HIFα isoforms (HIF1α, HIF2α and HIF3α) have been identified. Among these isoforms, HIF1α is the primary regulator of cellular responses to O₂ levels. HIF1α is an O₂ sensitive subunit, and its accumulation and degradation are highly sensitive to changing O₂ levels. When the O₂ level increases, HIF prolyl hydroxylases (HIF-PH) are activated and HIF1α is rapidly degraded. In a low O₂ environment, HIF-PH activity is suppressed, thereby increasing the steady-state levels of HIF1α. Then HIF1α can translocate to the nucleus, form a heterodimer with HIF1β, and activate the transcription of numerous genes and pathways involved in cell proliferation, cell differentiation, tissue regeneration and lipid metabolism. HIF2α share similarities with HIF1α, but also has its distinct function in modulating cell function. The function of HIF3α is currently unclear. Roxadustat belongs to the family of HIF prolyl hydroxylases inhibitors (HIF-PHI), which mimics the effect of low O₂ by increasing the levels of hypoxia-inducible factors. The information for human HIF1α (including isoform sequences) may be found at the World Wide Web site at uniprot.org/uniprot/Q16665. For example, the human HIF1α, isoform 1 comprises an amino acid sequence of

MEGAGGANDKKKISSERRKEKSRDAARSRRSKESEVFYELAHQLPLPHN
VSSHLDKASVMRLTISYLRVRKLLDAGDLDIEDDMKAQMNCFYLKALDG
FVMVLTDDGDMIYISDNVNKYMGLTQFELTGHSVFDFTHPCDHEEMREM
LTHRNGLVKKGKEQNTQRSFFLRMKCTLTSRGRTMNIKSATWKVLHCTG
HIHVYDTNSNQPQCGYKKPPMTCLVLICEPIPHPSNIEIPLDSKTFLSR
HSLDMKFSYCDERITELMGYEPEELLGRSIYEYYHALDSDHLTKTHHDM
FTKGQVTTGQYRMLAKRGGYVWVETQATVIYNTKNSQPQCIVCVNYVVS
GIIQHDLIFSLQQTECVLKPVESSDMKMTQLFTKVESEDTSSLFDKLKK
EPDALTLLAPAAGDTIISLDFGSNDTETDDQQLEEVPLYNDVMLPSPNE
KLQNINLAMSPLPTAETPKPLRSSADPALNQEVALKLEPNPESLELSFT
MPQIQDQTPSPSDGSTRQSSPEPNSPSEYCFYVDSDMVNEFKLELVEKL
FAEDTEAKNPFSTQDTDLDLEMLAPYIPMDDDFQLRSFDQLSPLESSSA
SPESASPQSTVTVFQQTQIQEPTANATTTTATTDELKTVTKDRMEDIKI
LIASPSPTHIHKETTSATSSPYRDTQSRTASPNRAGKGVIEQTEKSHPR
SPNVLSVALSQRTTVPEEELNPKILALQNAQRKRKMEHDGSLFQAVGIG
TLLQQPDDHAATTSLSWKRVKGCKSSEQNGMEQKTIILIPSDLACRLLG
QSMDESGLPQLTSYDCEVNAPIQGSRNLLQGEELLRALDQVN (SEQ
ID NO: 1; NCBI REFERENCE SEQUENCE: NP_001521.1)

The information for human HIF2α (a.k.a., Endothelial PAS domain-containing protein 1, or EPAS1) may be found at the World Wide Web site at uniprot.org/uniprot/Q99814. For example, the human HIF2α comprises an amino acid sequence of

MTADKEKKRSSSERRKEKSRDAARCRRSKETEVFYELAHELPLPHSVSS
HLDKASIMRLAISFLRTHKLLSSVCSENESEAEADQQMDNLYLKALEGF
IAVVTQDGDMIFLSENISKFMGLTQVELTGHSIFDFTHPCDHEEIRENL
SLKNGSGFGKKSKDMSTERDFFMRMKCTVTNRGRTVNLKSATWKVLHCT
GQVKVYNNCPPHNSLCGYKEPLLSCLIIMCEPIQHPSHMDIPLDSKTFL
SRHSMDMKFTYCDDRITELIGYHPEELLGRSAYEFYHALDSENMTKSHQ
NLCTKGQVVSGQYRMLAKHGGYVWLETQGTVIYNPRNLQPQCIMCVNYV
LSEIEKNDVVFSMDQTESLFKPHLMAMNSIFDSSGKGAVSEKSNFLFTK
LKEEPEELAQLAPTPGDAIISLDFGNQNFEESSAYGKAILPPSQPWATE
LRSHSTQSEAGSLPAFTVPQAAAPGSTTPSATSSSSSCSTPNSPEDYYT
SLDNDLKIEVIEKLFAMDTEAKDQCSTQTDFNELDLETLAPYIPMDGED
FQLSPICPEERLLAENPQSTPQHCFSAMTNIFQPLAPVAPHSPFLLDKF
QQQLESKKTEPEHRPMSSIFFDAGSKASLPPCCGQASTPLSSMGGRSNT
QWPPDPPLHFGPTKWAVGDQRTEFLGAAPLGPPVSPPHVSTFKTRSAKG
FGARGPDVLSPAMVALSNKLKLKRQLEYEEQAFQDLSGGDPPGGSTSHL
MWKRMKNLRGGSCPLMPDKPLSANVPNDKFTQNPMRGLGHPLRHLPLPQ
PPSAISPGENSKSRFPPQCYATQYQDYSLSSAHKVSGMASRLLGPSFES
YLLPELTRYDCEVNVPVLGSSTLLQGGDLLRALDQAT (SEQ ID
NO: 2; NCBI Reference Sequence: NP_001421.2)

The information for human HIF3α (including isoform sequences) may be found at the World Wide Web site at uniprot.org/uniprot/Q9Y2N7. For example, the human HIF3α, isoform 1 comprises an amino acid sequence of

MALGLQRARSTTELRKEKSRDAARSRRSQETEVLYQLAHTLPFARGVSA
HLDKASIMRLTISYLRMHRLCAAGEWNQVGAGGEPLDACYLKALEGFVM
VLTAEGDMAYLSENVSKHLGLSQLELIGHSIFDFIHPCDQEELQDALTP
QQTLSRRKVEAPTERCFSLRMKSTLTSRGRTLNLKAATWKVLNCSGHMR
AYKPPAQTSPAGSPDSEPPLQCLVLICEAIPHPGSLEPPLGRGAFLSRH
SLDMKFTYCDDRIAEVAGYSPDDLIGCSAYEYIHALDSDAVSKSIHTLL
SKGQAVTGQYRFLARSGGYLWTQTQATVVSGGRGPQSESIVCVHFLISQ
VEETGVVLSLEQTEQHSRRPIQRGAPSQKDTPNPGDSLDTPGPRILAFL
HPPSLSEAALAADPRRFCSPDLRRLLGPILDGASVAATPSTPLATRHPQ
SPLSADLPDELPVGTENVHRLFTSGKDTEAVETDLDIAQDADALDLEML
APYISMDDDFQLNASEQLPRAYHRPLGAVPRPRARSFHGLSPPALEPSL
LPRWGSDPRLSCSSPSRGDPSASSPMAGARKRTLAQSSEDEDEGVELLG
VRPPKRSPSPEHENFLLFPLSLSFLLTGGPAPGSLQDPSTPLLNLNEPL
GLGPSLLSPYSDEDTTQPGGPFQPRAGSAQAD (SEQ ID NO: 3;
NCBI Reference Sequence: NP_690008.2)

Multiple methods may be used to lower oxygen concentration in a subject, e.g., in the environment of the MG, such as near the eyes and/or eyelids area. For example, restricting of the blood flow can be accomplished in a number of ways, including by contracting or closing one or more blood vessels around the one or more dysfunctional MGs. For example, restricting the blood flow can be achieved, among other approaches, using one or more of a 532-nm potassium titanyl phosphate (KTP) laser, a 532-nm neodymium yttrium-aluminum-garnet (Nd:YAG) laser, a 578-nm copper vapor laser, 585-600-nm pulsed dye laser (PDL), a dual 595-nm PDL, a long-pulse alexandrite (755 nm), a 800-983-nm diode laser, a 1,064-nm Nd:YAG laser, indocyanine green augmented laser therapy, PDL treatment combined with rapamycin, intense pulsed light (IPL), carbon dioxide (CO₂) laser, cryotherapy, vascular endothelial growth factor (VEGF)/vascular endothelial growth factor receptor (VEGFR) inhibitors or antagonists, systemic and/or local beta-blockers, anti-angiogenic molecules and mixtures thereof. Any of these devices can be configured specifically for this treatment. The hypoxic status can be induced in one or more of the following ways: pharmaceutically, surgically, using a laser, using an intense-pulsed light, with a device and/or using hypoxia chamber goggles.

The effects of reduced oxygen concentration can also be mimicked by the systemic or local use of the agents described herein that induce the generation of HIFs in the dysfunctional MGs. These low oxygen mimetic agents include such drugs as one or more of prolyl hydroxylases inhibitors, i.e. FG-4592/roxadustat (Roxa), FG-2216, daprodustat/GSK1278863, vadadustat/AKB-6548, molidustat/BAY 85-3934, desidustat/ZYAN1), Dimethyloxalylglycine (DMOG), desferrioxamine (DFX) and cobalt chloride (CoCl₂), etc.

In some embodiments, restricting of the blood flow is accomplished by contracting or closing one or more blood vessels around the one or more dysfunctional meibomian glands. In some embodiments, restricting the blood flow is achieved, among other approaches, using one or more of a 532-nm potassium titanyl phosphate (KTP) laser, a 532-nm neodymium yttrium-aluminum-garnet (Nd:YAG) laser, a 578-nm copper vapor laser, 585-600-nm pulsed dye laser (PDL), a dual 595-nm PDL, a long-pulse alexandrite (755 nm), a 800-983-nm diode laser, a 1,064-nm Nd:YAG laser, indocyanine green augmented laser therapy, PDL treatment combined with rapamycin, intense pulsed light (IPL), carbon dioxide (CO₂) laser, cryotherapy, vascular endothelial growth factor (VEGF)/vascular endothelial growth factor receptor (VEGFR) inhibitors or antagonists, systemic and/or local beta-blockers, anti-angiogenic molecules and mixtures thereof. In some embodiments, the hypoxic status is induced pharmaceutically. In some embodiments, the hypoxic status is induced surgically. In some embodiments, the hypoxic status is induced using a laser. In some embodiments, the hypoxic status is induced using an intense-pulsed light. In some embodiments, the hypoxic status is induced with a device, such as hypoxia chamber goggles.

It was recently found that the oxygen-sensitive protein HIF1α plays an essential role in the regulation of human meibomian gland epithelial cells (HMGECs). To be specific, HIF1α activation promotes HMGEC differentiation. Although exposure to a low O₂ environment is a widely used method to stimulate HIF1α, systemic and prolonged hypoxia can lead to a range of severe pathophysiological reactions and compensations in vivo and will hinder its translation into clinical treatment in the future. In addition to a hypoxic environment, HIF1α expression also can be induced by low oxygen mimetic agents and growth factors. In order to mimic a local hypoxic environment around the MG, but also to minimize the compensatory effects of systemic low oxygen exposure, agents capable of pharmacological activation of the HIF1α pathway under normoxic (21% O₂) conditions were studied. It was found that the hypoxia mimetic Roxadustat (Roxa; also called FG-4592) can duplicate the differentiation-promoting effect of low oxygen by promoting HIF1α in immortalized HMGECs under normoxic condition. Roxa has been used tested for safety in animal studies and has passed the phase III clinical trial for the treatment of anemia in patients with chronic kidney disease.

### Combinational Therapy

In some embodiments, combinations of the low oxygen mimetic agents and the method to induce low oxygen concentration in the environment of MG, both described herein, are used. As described in the Examples, synergy for such combinational therapy was discovered. The agents and the method may be used simultaneously or sequentially.

### The Ability to Increase MG Function

The mechanisms underlying the low oxygen mimetic agents and/or the method/therapy of lowering oxygen concentration described herein were studied in the MGECs and an in vivo animal model. With no intention to be limiting, the agents described herein, such as Roxa, and/or a low oxygen concentration, such as no more than 1% O₂, increases expression levels and/or stability of at least one hypoxia-inducible factors (HIFs), such as HIF1α, in the MGECs. Other functions of the agents and the low oxygen method/therapy are described in the above sections.

### Decrease of Cholesterol Levels

The mechanisms underlying the low oxygen mimetic agents and/or the method/therapy of lowering oxygen concentration described herein were studied in the MGECs and an in vivo animal model. With no intention to be limiting, the agents described herein, such as Roxa, and/or a low oxygen concentration, such as no more than 1% O₂, reduces serum cholesterol levels in the treated subject. For example, the low oxygen mimetic agents and/or the method/therapy of lowering oxygen concentration described herein reduces cholesterol levels (e.g., serum cholesterol levels) in the treated subject to about 90%, 80%, 70%, 60%, 50%, 40%, 30%, 20%, 10%, or less, compared to a subject with a same disease or disorder without the treatment, or a healthy subject. The cholesterol levels in a subject may be measured using standard blood test or other methods known in the art, and may include indexes including at least one of total cholesterol amount [a measure of the total amount of cholesterol in blood, including both low-density lipoprotein (LDL) cholesterol and high-density lipoprotein (HDL) cholesterol], LDL cholesterol amount (the main source of cholesterol buildup and blockage in the arteries), HDL cholesterol amount (HDL helps remove cholesterol from arteries), Non-HDL amount [the total cholesterol minus HDL, including only LDL and other types of cholesterol such as VLDL (very-low-density lipoprotein)], triglycerides amount, etc. In some embodiments, the low oxygen mimetic agents and/or the method/therapy of lowering oxygen concentration described herein reduces the levels of at least one of the total cholesterol amount, the LDL cholesterol amount, the Non-HDL amount, and the triglycerides amount. In some embodiments, the hypoxia-mimetic agents and/or the method/therapy of lowering oxygen concentration described herein reduces the levels of at least one of the total cholesterol amount, the LDL cholesterol amount, the Non-HDL amount, and the triglycerides amount in the treated subject to about 90%, 80%, 70%, 60%, 50%, 40%, 30%, 20%, 10%, or less, compared to a subject with a same disease or disorder without the treatment, or a healthy subject. In some embodiments, the low oxygen mimetic agents and/or the method/therapy of lowering oxygen concentration described herein increases the levels of the HDL cholesterol amount in the treated subject to about 10%, 20%, 30%, 40%, 50%, 60%, 70%, 80%, 90%, 100%, 150%, 200%, 300%, 500%, 1000%, or more, than the levels in a subject with a same disease or disorder without the treatment, or a healthy subject.

The terms “normal cholesterol levels” and “normal serum cholesterol levels” refers to levels in a common healthy subject or a subject without a specific disease or disorder described herein, recognized by a doctor or a medical personnel. For example, healthy serum cholesterol may be less than 200 mg/dL, healthy LDL cholesterol may be less than 130 mg/dL, healthy HDL cholesterol may be higher than 55 mg/dL for women and 45 mg/dL for men, and healthy triglycerides may be less than 150 mg/dL. Normal cholesterol levels may refer to a condition when any of these levels are in the healthy levels. High cholesterol levels may refer to a condition when any of these levels are out of the healthy levels. For example, high cholesterol levels may refer to at least 10%, 20%, 30%, 40%, 50%, 60%, 70%, 80%, 90%, 100%, 150%, 200%, 300%, 500%, 1000%, or more, levels than healthy levels for serum cholesterol, LDL, and/or triglycerides, and/or at least 10%, 20%, 30%, 40%, 50%, 60%, 70%, 80%, 90%, or less, levels than healthy levels for HDL. For example, normal cholesterol levels may refer to at most 30%, 25%, 20%, 15%, 10%, 5%, or less, more than healthy levels for serum cholesterol, LDL, and/or triglycerides, and at most 5%, 10%, 15%, 20%, 25%, 30%, or more, less than healthy levels for HDL.

### Diseases and Disorders

The diseases and disorders related to reduced MG functions, as a target for the agents and/or the low oxygen treatment described herein, comprises at least one selected from the group consisting of: MG dysfunction (MGD), dry eye disease (DED), Sjogren's syndrome, systemic lupus erythematosus, rheumatoid arthritis, etc.

According to a summary of from the Tear Film and Ocular Surface Society (see Nichols et al. Invest Ophthalmol Vis Sci. 2011 52:1922-1929), Meibomian gland dysfunction (MGD) is a chronic, diffuse abnormality of the meibomian glands, commonly characterized by terminal duct obstruction and/or qualitative/quantitative changes in the glandular secretion. It may result in alteration of the tear film, symptoms of eye irritation, clinically apparent inflammation, and ocular surface disease. The major risk factors of MGD include aging, androgen deficiency, stem cell imbalance, and retinoic acid (RA) use. These factors lead to disrupted differentiation and renewal of meibocytes, obstruction of meibomian orifices, stasis of meibum, acinar atrophy, gland dropout and eventually to MGD. Meibomian gland disease is used to describe a broader range of meibomian gland disorders, including neoplasia and congenital disease. Other terms such as meibomitis or meibomianitis describe a subset of disorders of MGD associated with inflammation of the meibomian glands. Although inflammation may be important in the classification and in the therapy of MGD, these terms are not sufficiently general, as inflammation is not always present. There is no global cure for MGD. Improvement in the patient's symptoms is the major goal in the treatment of MGD.

A classification of MGD into two major categories based on meibomian gland secretion is proposed: low-delivery states and high-delivery states. Low-delivery states are further classified as hyposecretory or obstructive, with cicatricial and noncicatricial subcategories. Hyposecretory MGD describes the condition of decreased meibum delivery due to abnormalities in meibomian glands without remarkable obstruction. Obstructive MGD is due to terminal duct obstruction. In the cicatricial form, the duct orifices are dragged posteriorly into the mucosa, whereas these orifices remain in their normal positions in noncicatricial MGD. High-delivery, hypersecretory MGD is characterized by the release of a large volume of lipid at the lid margin that becomes visible on application of pressure onto the tarsus during examination. Each MGD category also has primary causes, referring to conditions for which there are no discernible underlying causes or etiology.

In some embodiments, a subject to be treated has a diseases or disorder related to increased serum cholesterols, and/or increased weight gain. For example, the subject may have obesity, diabetes, overweight or other related diseases or disorders.

Diagnostic methods for the diseases or disorders to be treated by the low oxygen mimetic agents and/or the method/therapy to reduce local oxygen concentration in MG, as described herein, are well known in the art. For example, Sjogren's syndrome is a complex and currently incurable autoimmune disorder, which is characterized by dry eyes and a dry mouth. This condition often accompanies other immune system disorders, such systemic lupus erythematosus or rheumatoid arthritis. These diseases cause decreased aqueous tear production, leading to aqueous-deficient dry eye disease (DED). They are also associated with meibomian gland dysfunction (MGD) and evaporative DED.

Exemplary diagnosis methods for MGD are known in the art. See Rolando and Vagge (2014) Current Ophthalmology Reports volume 2, pages 65-74 or Nichols et al. (2011) Invest Ophthalmol Vis Sci. 52:1922-1929. A summary of specialized and nonspecialized tests for MGD and MGD-related disease is given below, adapted from Nichols et al. (2011).

Specialized and Nonspecialized Tests for MGD and MGD-Related Disease

| Testing | Specific | Tests for a | Tests for a |
|---|---|---|---|
| Category | Test(s) | General Clinic | Specialized Unit |
| Symptoms | Questionnaires | McMonnies; | McMonnies; Schein; |
| | | Schein; ODSI; | ODSI; DEQ; OCI; |
| | | DEQ; OCI; | SPEED; and others |
| | | SPEED; and | |
| | | others | |
| Signs | | | |
| Meibomian | Lid | Slit lamp | Slit lamp microscopy; |
| function | morphology | microscopy | confocal microscopy |
| | Meibomian | | Meibography |
| | gland mass | | |
| | Gland | Slit lamp | Slit lamp microscopy |
| | expressibility; | microscopy | |
| | expressed oil | | |
| | quality and | | |
| | volume | | |
| | Lid margin | | Meibometry |
| | reservoir | | |
| | Tear film lipid | Interferometry, | Interferometry; slit |
| | layer; | slit lamp | lamp; video |
| | thickness, | | interferometry |
| | spread time, | | |
| | spread rate | | |
| Evaporation | Evaporimetry | | Evaporimetry |
| Tears | | | |
| Osmolarity | Osmolarity | TearLab device, | TearLab device, other |
| | | other | |
| Stability | Tear film | TFBUT; Ocular | TFBUT; Ocular |
| | | protection index | protection index |
| | Tear film lipid | Spread time | Interferometry; spread |
| | layer | | rate; pattern |
| Indices of | Tear secretion | Schimer 1 | Fluorophotometry/ |
| volume and | | | fluorescetin |
| secretion | | | clearance rate |
| | Tear volume | Not available | Volume by |
| | | | fluorophotometry |
| | Tear volume | Meniscus height | Meniscus radius of |
| | | | curvature; meniscometry |
| | Tear clearance | Tear film index | Tear film index |
| Oscular | Oscular surface | Oxford scheme; | Oxford scheme; |
| surface | staining | NEI/industry | NEI/industry scheme |
| Inflammation | Biomarkers | scheme | Flow cytometry; bead |
| | | | arrays; microarrays; mass |
| | | | spectrometry; cyrokines |
| | | | and other mediators; |
| | | | interleukins; matrix |
| | | | metalloproteinases |

Tests of glandular functions are presented first followed by those for related disorders such as dry eye.
OSDI, Oscular Surface Disease Inders;
DEQ, Dry Eye Questionaire;
OCI, Oscular Comfort Inders;
SPEED, Standard Patient Evaluation of Eye Dryness.

There is no practicable way to measure O₂ level or the activity of HIF proteins in human meibomian gland directly. However, other techniques known in the art, e.g., positron emission tomography (PET) may be used to measure the aerobic glycolysis, as an indirect indicator.

Exemplary outcome measurements for the improvement of MGD and DED by the low oxygen mimetic agents and/or the method/therapy to reduce local oxygen concentration may include: increased tear secretion and tear volume, prolonged tear film break up time, improved tear osmolarity, improved gland expressibility and expressed oil quality and volume, increased thickness of tear film lipid layer, decreased ocular surface staining, decreased inflammation on the ocular surface, and alleviated discomfort feeling of patients. For example, Meibum quality is assessed in each of eight glands of the central third of the lower lid on a scale of 0 to 3 for each gland: 0, clear; 1, cloudy; 2, cloudy with debris (granular); and 3, thick, like toothpaste (total score range, 0-24). Expressibility is assessed on a scale of 0 to 3 in five glands in the lower or upper lid, according to the number of glands expressible: 0, all glands; 1, three to four glands; 2, one to two glands; and 3, no glands. Staining scores are obtained by summing the scores of the exposed cornea and conjunctiva. Oxford staining score range, 1-15; DEWS staining score range, 0-33. In some embodiments, the therapy and/or the low oxygen mimetic agents, described herein, improve the Meibum quality in at least one of measurable indexes described herein.

Tear volume and tear secretion may be measured by fluorophotometric examinations. For example, a 0.1% fluorescein solution (1 μl) may be applied to the lateral upper bulbar conjunctiva and mixed with the tear film. Ten seconds after application, the clearance of tear film fluorescein may be determined by measuring the decrease in tear film fluorescein five times with 1 minute intervals, using a Fluorotron Master (Coherent Radiation Inc). A regression line for the clearance of the tear film fluorescein and the tear film turnover rate may be automatically calculated (Eter and Göbbels, Br J Ophthalmol. 2002 86:616-619). Similar tests include Schirmer's test, which determines whether the eye produces enough tears to keep it moist. In some embodiments, the therapy and/or the low oxygen mimetic agents, described herein, improve the tear volume and/or tear secretion of the treated subject to about 10%, 20%, 30%, 40%, 50%, 60%, 70%, 80%, 90%, 100%, 150%, 200%, 250%, 300%, 500%, 1000%, or more.

Tear breakup time (TBUT) is a clinical test used to assess for evaporative dry eye disease. To measure TBUT, fluorescein is instilled into the patient's tear film and the patient is asked not to blink while the tear film is observed under a broad beam of cobalt blue illumination. The TBUT is recorded as the number of seconds that elapse between the last blink and the appearance of the first dry spot in the tear film, as seen in this progression of these slit lamps photos over time. A TBUT under 10 seconds is considered abnormal. This patient also has punctate epithelial erosions (PEE) that stain positively with fluorescein, another sign of ocular surface dryness. TBUT measurement is an easy and fast method used to assess the stability of tear film. In some embodiments, the therapy and/or the low oxygen mimetic agents, described herein, improve the tear breakup time (TBUT) of the treated subject to about 10%, 20%, 30%, 40%, 50%, 60%, 70%, 80%, 90%, 100%, 150%, 200%, 250%, 300%, 500%, 1000%, or more.

One of the principal indicators of tear dysfunction is elevated tear film osmolarity (hyperosmolarity), predominantly due to elevated sodium ion concentration. Elevated osmolarity is considered the central mechanism of ocular surface damage and may be the single best marker for dry eye disease. The diagnostic cut-off is in excess of 316 mOsmol/L. In some embodiments, the therapy and/or the low oxygen mimetic agents, described herein, decreases the tear osmolarity of the treated subject to about 90%, 80%, 70%, 60%, 50%, 40%, 30%, 20%, 10%, or less 10%. It was reported that an average tear osmolarity is of 302±9.7 in normal subjects (815) and is of 326.9±22.1 in subjects with keratoconjunctivitis sicca (621). In some embodiments, the therapy and/or the low oxygen mimetic agents, described herein, decreases the tear osmolarity of the treated subject to about 315, 314, 313, 312, 311, 310, 309, 308, 307, 306, 305, 304, 303, 302, 301, 300, 299, 298, 297, 296, 295, 294, 293, 292, 291, 290 mOsmol/L, or less.

### Obesity

Obesity is a medical condition in which excess body fat has accumulated to an extent that it may have a negative effect on health. People are generally considered obese when their body mass index (BMI), a measurement obtained by dividing a person's weight by the square of the person's height, is over, e.g., 30 kg/m²; the range 25-30 kg/m² is usually defined as overweight. In adults, obesity is defined as having a BMI of 30.0 or more, according to the Centers for Disease Control and Prevention (CDC). The relationship between adult BMI and obesity class is: BMI of 18.5 or under is considered underweight, 18.5 to <25.0 is considered “normal” weight, 25.0 to <30.0 is considered overweight, 30.0 to <35.0 is considered class 1 obesity, 35.0 to <40.0 is considered class 2 obesity, 40.0 or over is considered class 3 obesity (also known as morbid, extreme, or severe obesity). For a doctor to diagnose a child over 2 years old or a teen with obesity, their BMI has to be in the 95th percentile for people of their same age and biological sex as below: >5% percentile of BMI is considered underweight, 5% to <85% is considered “normal” weight, 85% to <95% is considered overweight, 95% or over is considered obesity.

Some East Asian countries use lower values. Obesity increases the likelihood of various diseases and conditions, particularly cardiovascular diseases, type 2 diabetes, obstructive sleep apnea, certain types of cancer, osteoarthritis, and depression.

The symptoms of obesity may include: Above average body weight, Trouble sleeping, Sleep apnea—a condition in which breathing is irregular and periodically stops during sleep, Varicose veins, Skin problems caused by moisture that accumulates in the folds of your skin, Gallstones, Osteoarthritis in weight-bearing joints, especially the knees.

BMI is a rough calculation of a person's weight in relation to their height. Other more accurate measures of body fat and body fat distribution include: skinfold thickness tests, waist-to-hip comparisons, screening tests, such as ultrasounds, CT scans, and MRI scans. Other tests to help diagnose obesity-related health risks may include: blood tests to examine cholesterol and glucose levels, liver function tests, a diabetes screening, thyroid tests, heart tests, such as an electrocardiogram (ECG or EKG).

### Ocular Surface Disease Index (OSDI)

The Ocular Surface Disease Index (OSDI) is a 12-item questionnaire that provides a rapid assessment of the symptoms of ocular irritation consistent with ocular surface disease, including posterior blepharitis and dry eye disease, and their impact on vision-related functioning. The 12 items of the OSDI questionnaire are graded on a scale of 0 to 4, where 0 indicates none of the time; 1, some of the time; 2, half of the time; 3, most of the time; and 4, all of the time. The total OSDI score is then calculated on the basis of the following formula: OSDI=[(sum of scores for all questions answered)×100]/[(total number of questions answered)×4]. Thus, the OSDI is scored on a scale of 0 to 100, with higher scores representing greater disability. A negative change from baseline indicates an improvement in vision-related function and the ocular inflammatory disorders described herein. For the therapeutic method described herein, treatment is considered more effective than control (vehicle) as indicated by a mean change (decrease) from baseline for the OSDI of >10 units compared to control.

Therapeutic treatment is considered more effective than the vehicle as indicated by a mean change from baseline of average score (0-100) for the Ocular Surface Disease Index (OSDI) of >10 units better than vehicle.

### Tear Film Break-Up Time

The standard TBUT measurement is performed by moistening a fluorescein strip with sterile non-preserved saline and applying it to the inferior tarsal conjunctiva. After several blinks, the tear film is examined using a broad beam of the slit lamp with a blue filter. The time lapse between the last blink and the appearance of the first randomly distributed dark discontinuity in the fluorescein stained tear film is measured three times and the mean value of the measurements is calculated. The tear break-up time is evaluated prior to the instillation of any eye drops and before the eyelids are manipulated in any way. Break-up times less than 10 seconds are considered abnormal. A positive change from baseline indicates improvement in symptoms of the ocular inflammatory disorders described herein. The treatment described herein, leads to an improvement in TBUT significantly greater than that observed from treatment with vehicle alone.

### Corneal and Conjunctival Staining

Corneal staining is a measure of epithelial disease, or break in the epithelial barrier of the ocular surface, typically seen with ocular surface disorders such as posterior blepharitis and dry eye, among others. Importantly, corneal staining can exist even without clinically evident dry eye, if there is significant lid disease, such as posterior blepharitis. Corneal staining is highly correlated with ocular discomfort in many, though not all patients; in general corneal staining is associated with high scores in the OSDI, as described above. For corneal fluorescein staining, saline-moistened fluorescein strips or 1% sodium fluorescein solution are used to stain the tear film. The entire cornea is then examined using slit-lamp evaluation with a yellow barrier filter (#12 Wratten) and cobalt blue illumination (staining is more intense when it is observed with a yellow filter). Staining is graded according to the Oxford Schema.

Conjunctival staining is a measure of epithelial disease or break in the epithelial barrier of the ocular surface, typically seen with ocular surface disorders such as posterior blepharitis and dry eye, among others. Importantly, conjunctival staining, similar to corneal staining, can exist even without clinically evident dry eye, if there is significant lid disease, such as posterior blepharitis. Conjunctival staining can also correlate with symptoms of ocular irritation and high OSDI scores as described above. Conjunctival staining is performed under the slit-lamp using lissamine green. Saline-moistened strip or 1% lissamine green solution is used to stain the tear film, and interpalpebral conjunctival staining is evaluated more than 30 seconds, but less than 2 minutes, later. Using white light of moderate intensity, only the interpalpebral region of the nasal and temporal conjunctival staining is graded using the Oxford Schema (above). The treatment described herein leads to decreases in ocular staining scores beyond what is observed with the vehicle alone.

Therapeutic treatment is considered more effective than vehicle as indicated by a mean change from baseline in average score (0-5 scale) for corneal and conjunctival staining of >1 unit better than vehicle, e.g. as detected using the Oxford Schema.

### Schirmer Test

The Schirmer test is performed in the presence and in the absence of anesthesia by placing a narrow filter-paper strip (5×3 5 mm strip of Whatman #41 filter paper) in the inferior cul-de-sac. This test is conducted in a dimly lit room. The patient gently closes his/her eyes until five minutes have elapsed and the strips are removed. Because the tear front will continue advancing a few millimeters after it has been removed from the eyes, the tear front is marked with a ball-point pen at precisely five minutes. Aqueous tear production is measured by the length in millimeters that the strip wets during 5 minutes. Results of 10 mm or less for the Schirmer test without anesthesia and 5 mm or less for the Schirmer test with anesthesia are considered abnormal. A positive change from baseline indicates improvement of one or more symptoms of an ocular inflammatory disorder described herein.

### Meibomian Gland Evaluation

In the center of the lower lid, 10 adjacent central glands are located on both sides and the glands are expressed by applying a firm digital pressure at the base of the glands. The number of glands expressed for each eye is documented. The quality of secretion is described as follows:
Clear excreta or clear with small particles (0)
Opaque excreta with normal viscosity (1)
Opaque excreta with increased viscosity (2)
Secretions retain shape after expression (3)

Posterior blepharitis is associated with lid inflammation and alterations in the quantity and/or quality of the meibomian gland secretions, with severe disease associated with quality grades 2-3, as described above. The treatment described herein leads to improvement in meibomian secretion characterized by a decrease in this score; for example, from 3 to 2, or from 2 to 1. An improvement is indicated by a mean change from baseline (0-3 scale) for meibomian gland secretion quality of >1 unit better than vehicle.

### Lid and Lid Margin Erythema

Lid margin vascular injection (erythema) is defined as a red discoloration, compared to the surrounding eyelid skin and is graded as follows:

| | | |
|---|---|---|
| None | (0): | none |
| Mild | (1): | redness localized to a small region of the lid |
| | | margin(s) or skin |
| Moderate | (2): | redness of most of the lid margin(s) |
| Severe | (3): | redness of most or all the lid margin(s) and skin |
| Very Severe | (4): | marked diffuse redness of both lid margins and skin |

The presence or absence of tarsal telangiectasis is also noted. Lid telangiectasia is defined as the presence of at least two blood vessels along the eyelid margin.

### Conjunctiva Hyperemia

Bulbar conjunctival hyperemia is graded as follows:

| | | |
|---|---|---|
| None | (0): | none |
| Mild | (1): | slight localized injection |
| Moderate | (2): | pink color, confined to palpebral or bulbar |
| | | conjunctiva |
| Severe | (3): | red color of the palpebral and/or bulbar conjunctiva |
| Very Severe | (4): | marked dark redness of the palpebral and/or bulbar |
| | | conjunctiva |

The presence or absence of tarsal papillary hypertrophy is also noted.

### Formulations and Dosage for Administration

Formulations of the present invention include those suitable for oral, nasal, topical, transdermal, buccal, sublingual, intramuscular, intracardiac, intraperotineal, intrathecal, intracranial, rectal, vaginal and/or other parenteral (e.g., intravenous) administration of the agents described herein, e.g., Roxa. The formulations may conveniently be presented in unit dosage form and may be prepared by any methods well known in the art of pharmacy. The amount of active ingredient, e.g., Roxa, which can be combined with a carrier material to produce a single dosage form, will generally be that amount of the compound that produces a therapeutic effect.

Pharmaceutically available and/or effective administration routes may be used to deliver the agents (such as Roxa) described herein to a subject. With no intention to be limiting, the agents may be administered locally or systemically to a subject, including administering to, e.g., the circulatory system and/or the GI tract (the digestive system) of the subject, via, e.g., inhalation, pulmonary lavage, oral ingestion, anal administration, infusion, and/or injection, or to the local area near the eyes or the eyelids (e.g., through topical administration). Administration routes also include, but not limited to, administering to a subject intravenously, intradermally, intraperitoneally, intrapleurally, intratracheally, intramuscularly, subcutaneously, by injection, and by infusion. In some embodiments, the administration routes include oral and/or intravenous administration. In some embodiments, the administration routes comprise local injection, such as subconjunctiva injection, subdermal injection around the eyelids, or periorbital injection.

Dosages and dosing regimens may be determined by a doctor or a medical personnel accordingly to each patient's individual situation. Generally, the agents (such as Roxa) can be given orally at about 1-20 g/day to improve MG functions and/or to treat a disease or disorder described herein. Oral dosage formulations may be administered either in tablet or powder form, or conveniently dissolved in a little water or another beverage, or included in foodstuffs or in food or medical supplements. The preferred adult dose of the agents (such as Roxa) is generally equivalent to about 10 g per day, but anywhere between about 0.1 g per day up to about 20 g or about 30 g per day may be taken. For example, potential dosages may include about 0.01 g to about 30 g per day, about 0.01 g to about 30 g per day, about 0.1 g to about 30 g per day, about 1 g to about 30 g per day, about 1 g to about 5 g per day, about 5 g to about 10 g per day, or any dosages found effective to the subject. Specifically, potential dosages may include about 0.01 to about 200 mg/kg body weight, about 0.01 to about 100 mg/kg body weight, about 0.05 to about 200 mg/kg body weight, about 0.05 to about 100 mg/kg body, weight, about 0.05 to about 50 mg/kg body weight, about 0.1 to about 200 mg/kg body weight, about 0.01 to about 100 mg/kg body weight, about 0.1 to about 50 mg/kg body weight, about 0.1 to about 20 mg/kg body weight, about 0.1 to about 10 mg/kg body weight, about 0.1 to about 5 mg/kg body weight, about 0.5 to about 200 mg/kg body weight, about 0.5 to about 100 mg/kg body weight, about 0.5 to about 50 mg/kg body weight, about 0.5 to about 20 mg/kg body weight, about 0.5 to about 10 mg/kg body weight, about 0.5 to about 5 mg/kg body weight, about 1 to about 200 mg/kg body weight, about 1 to about 100 mg/kg body weight, about 1 to about 50 mg/kg body weight, about 1 to about 30 mg/kg body weight, about 1 to about 20 mg/kg body weight, about 1 to about 10 mg/kg body weight, about 1 to about 5 mg/kg body weight, etc. In some embodiments, the agents (such as Roxa) is administered to the subject in a dosage regimen of about 0.1 to about 10 mg/kg body weight. The oral dosing of Roxa was 50-70 mg three times per week to treat anemia. Unless more proofs are available otherwise, similar doses should be used for treating MGD and DED (and other diseases or disorders described herein). In some embodiments, the dose for treating obesity is about 50-500 mg per time, for example, about 50-300 mg per time, about 50-200 mg per time, about 50-100 mg per time, about 100-500 mg per time, about 100-300 mg per time, about 100-200 mg per time, about 200-500 mg per time, about 200-300 mg per time, about 300-500 mg per time, etc. In some embodiments, the dose is about 1-300 μg per injection, for example, about 1-200 μg per injection, about 1-100 μg per injection, about 1-50 per injection, about 1-20 μg per injection, about 1-10 μg per injection, about 20-300 per injection, about 20-200 μg per injection, about 20-100 μg per injection, about 50-300 per injection, about 50-200 μg per injection, about 50-100 μg per injection, about 100-200 per injection, about 100-300 μg per injection, about 200-300 μg per injection, etc. In some embodiments, the concentration of the agents (e.g., Roxa) in eye drops is about 0.01-100 mg/ml, for example, about 0.01 mg/ml to about 50 mg/ml, about 0.1 mg/ml to about 50 mg/ml, about 0.5 mg/ml to about 50 mg/ml, about 1 mg/ml to about 50 mg/ml, about 1 mg/ml to about 30 mg/ml, about 1 mg/ml to about 20 mg/ml, about 1 mg/ml to about 10 mg/ml, about 0.01 mg/ml to about 30 mg/ml, about 0.01 mg/ml to about 20 mg/ml, about 0.01 mg/ml to about 10 mg/ml, about 0.1 mg/ml to about 50 mg/ml, about 0.1 mg/ml to about 20 mg/ml, about 0.1 mg/ml to about 10 mg/ml, about 50 mg/ml to about 100 mg/ml, about 10 mg/ml to about 100 mg/ml, about 20 mg/ml to about 100 mg/ml, about 30 mg/ml to about 100 mg/ml, etc. With no intention to be limiting, the approved dose for Evrenzo® (roxadustat) Tablets to treat anemia associated with chronic kidney disease in dialysis patients is, for adults, 50 mg (for patients not on erythropoiesis-stimulating agent treatment) or 70 or 100 mg (for patients switching from erythropoiesis-stimulating agents) as the starting dose, orally administered three times weekly, while the thereafter dosages should be adjusted according to the patient's condition with a maximum dose not exceeding 3.0 mg/kg.

In some cases, the agents (such as Roxa) is administered twice or more, e.g., 3 times, 4 times, 5 times, 6 times, 7 times, 8 times, 9 times, 10 times, 15 times, 20 times, 25 times, 30 times, 35 times, 40 times, 50 times, 60 times, 70 times, 80 times, 90 times or more. For example, the agents (such as Roxa) is administered at least once per week, e.g., at least twice per week, at least three times per week, at least four times per week, at least five times per week, at least six times per week, at least seven times per week. Alternatively, the agents (such as Roxa) is administered at least once per day, e.g., at least twice per day, at least every eight hours, at least every four hours, at least every two hours, or at least every hour. The compositions of the invention (e.g., the dosage formulation of the agents, such as Roxa) are administered for a duration of 1 day, 2 days, 3 days, 4 days, 5 days, 6 days, 7 days, 2 weeks, 3 weeks, 4 weeks, five weeks, six weeks, 2 months, 3 months, 4 months, 5 months, 6 months, 7 months, 8 months, 9 months, 10 months, 11 months, 12 months, 2 years, 3 years, 4 years, 5 years or more. For example, the composition of the invention are administered one dose every two weeks for 4 to 6 weeks or until the cancer is treated.

Optionally, the subject is also treated with at least one additional agent and/or therapy (e.g., low oxygen treatment). In some cases, the agents (such as Roxa) described herein is administered simultaneously with the at least one additional agent and/or therapy (e.g., low oxygen treatment). Alternatively, the agents (such as Roxa) described herein and the at least one additional therapy (e.g., low oxygen treatment) are administered sequentially.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

The Role of Hypoxia-Inducible Factor 1α in the Regulation of Human Meibomian Gland (MG) Epithelial Cells

This Example describes an exemplary design and experimentation to analyze the HIF1α expression in the human MG under hypoxic environment or upon Roxa treatment. The effect of Roxa on stimulating differentiation in IHMGECs was also analyze.

Optimal meibomian gland (MG) function is critically important for the health and wellbeing of the ocular surface [Knop et al. (2011) Investigative Ophthalmology & Visual Science 52:1938-1978; Bron et al. (2017) The Ocular Surface 15:438-510]. These glands synthesize and secrete a proteinaceous lipid mixture (i.e. meibum) that enhances the stability and prevents the evaporation of the tear film [Knop et al. (2011); Bron et al. (2017); Green-Church et al. (2011) Investigative Ophthalmology & Visual Science 52:1979-1993]. MG dysfunction (MGD), in turn, leads to a loss of meibum, destabilization and hyperevaporation of the tear film, and dry eye disease (DED) [Knop et al. (2011); Bron et al. (2017); Green-Church et al. (2011); Bron and Tiffany (2004) The Ocular Surface 2:149-165]. Indeed, MGD is the most common cause of DED [Knop et al. (2011); Bron et al. (2017); Green-Church et al. (2011); Bron and Tiffany (2004); Nelson et al. (2011) Investigative Ophthalmology & Visual Science 52:1930-1937; Lemp et al. (2012) Cornea 31:472-478; Nichols et al. (2011) Investigative Ophthalmology & Visual Science 52:1922-1929].

There is no known cure for MGD. This fact is likely due to the relative lack of information concerning the physiological regulation of the human MG in health and disease. Recently, it was discovered that a hypoxic environment is beneficial for MG function [Liu et al. (2019)]. More specifically, it was found that human and mouse MGs exist in a hypoxic environment in vivo, and that low oxygen (O₂) stimulates differentiation of immortalized human meibomian gland epithelial cells (IHMGECs) in vitro. This hypoxic response involves an increased expression of sterol regulatory element binding protein 1 (SREBP1), an enlargement of lysosomes and a rise in deoxyribonuclease (DNase) II activity [Liu et al (2019)]. SREBP1 is a key controller of lipid synthesis [Horton et al. (2002) The Journal of Clinical Investigation 109:1125-1131], and DNase II is a biomarker for terminal differentiation and holocrine secretion [Zouboulis (2017) The Journal of Investigative Dermatology 137:537-539; Fischer et al. (2017) The Journal of Investigative Dermatology 137:587-594]. In contrast, the local hypoxic environment may be lost in MGD. The results suggest that re-induction of this hypoxic status may be a breath of fresh air for the treatment of MGD in future.

The mechanism(s) underlying this effect of hypoxia on the MG is unknown, but it is hypothesized that it is due to an increase in the levels of hypoxia-inducible factor1 α (HIF1α). HIF1a is the primary regulator of cellular responses to hypoxia [Wang et al. (1995) Proceedings of the National Academy of Sciences of the United States of America 92:5510-5514; Iyer et al. (1998) Genes & Development 12:149-162]. In other tissues HIF1α expression can be induced by multiple stimuli, including hypoxia, low oxygen mimetic agents, and certain growth factors [Coimbra et al. (2004) Osteoarthritis Cartilage 12:336-345; Stroka et al. (2]001) FASEB Journal 15:2445-2453; Nakada et al. (2017) Nature 541:222-227; Ferrari et al. (2017) Proceedings of the National Academy of Sciences of the United States of America 114:E4241-E4250; Jain et al. (2016) Science 352:54-61; Kim et al. (2018) Leukemia 32:2672-2684; Botusan et al. (2008) Proceedings of the National Academy of Sciences of the United States of America 105:19426-19431]. Typically, HIF1α is rapidly degraded, but exposure to hypoxic conditions [i.e. under 5% O₂; see Bracken et al. (2006) The Journal of biological chemistry 281:22575-22585] prevents degradation and stabilizes this protein. Once stabilized, HIF1a translocates to the nucleus and activates the transcription of numerous genes [Wenger and Gassmann (1997) Biol Chem 378:609-616], such as those promoting cell survival and proliferation, and glucose (e.g. glucose transporter 1) and lipid metabolism (e.g. SREBP1) [Liu et al. (2019); Wang et al. (1995); Stroka et al. (2001); Bensaad et al. (2014) Cell Rep 9:349-365; Lee et al. (2015) Stem Cells 33:2182-2195].

A common way to induce HIF1α expression, other than low O₂, is through the use of the low oxygen mimetic drugs Roxadustat (Roxa), dimethyloxalylglycine, desferrioxamine and cobalt (II) chloride (CoCl₂) [Jain et al. (2016) Science 352:54-61; Abdel-Rahman et al. (2019) Biomed Pharmacother 109:1688-1697; Ogle et al. (2012) Neurobiol Dis 45:733-742]. These compounds create a pseudohypoxic environment and initiate a HIF1α-mediated hypoxic response, albeit under normoxic conditions, both in vivo and in vitro [Hill et al. (2008) Journal of the American Society of Nephrology 19:39-46; Maybin et al. (2018) Nat Commun 9:295; Hoppe et al. (2016) Proceedings of the National Academy of Sciences of the United States of America 113:E2516-2525; Nguyen et al. (2013) Journal of Cell Science 126:1454-1463; Wang and Semenza (1993) Blood 82:3610-3615; Tian et al. (2011) The Journal of biological chemistry 286:13041-13051; Taheem et al. (2018) Stem Cells 36:1380-1392]. These drugs have been used to facilitate wound healing [Tang et al. (2018) Cellular physiology and biochemistry: international journal of experimental cellular physiology, biochemistry, and pharmacology 46:2460-2470], treat retinopathy of prematurity [Hoppe et al. (2016) Proceedings of the National Academy of Sciences of the United States of America 113:E2516-2525], protect corneal endothelial cells [Bhadange et al. (2018) Cornea 37:501-507], and regenerate tissues [Yu et al. (2018) Biomaterials 165:48-55; Heber-Katz (2017) Trends Mol Med 23:1024-1036].

This Example evaluated whether HIF1α is present in the human MG and determined whether exposure to 1% O₂ (hypoxia), or to Roxa treatment at 1% or 21% O₂ (normoxia), increases HIF1α expression in IHMGECs. It is further examined whether Roxa can mimic a hypoxic condition by stimulating differentiation in IHMGECs.

### Methods and Materials

### Human Tissues

Human eyelid tissues were collected after ectropion surgery from patients without MGD (1 male, 2 females, age range=70 to 82 years), and processed for frozen section preparation as previously described in Liu et al. (2019) The ocular surface 17(2):310-317. All samples were de-identified according to Health Insurance Portability and Accountability Act requirements. The use of human tissues was approved by the Institutional Review Board of the Massachusetts Eye and Ear Infirmary and Schepens Eye Research Institute and adhered to the tenets of the Declaration of Helsinki.

### Immunofluorescence Staining

Frozen sections of human MGs were incubated overnight at 4° C. in a moist chamber with an antibody specific for HIF1α (28b, 1:50, SC-13515, Santa Cruz Biotechnology, Dallas, Tex.). After three times of phosphate buffered saline rinses, the sections were incubated with donkey anti-mouse secondary antibody (1:200, 2492098, EMD Millipore, Temecula, Calif.) for 1 hour at room temperature. The primary antibody was replaced with mouse IgG (Cell Signaling Technology, Danvers, Mass.) for negative controls. All slides were mounted with ProLong Gold antifade containing 4′,6-diamidino-2-phenylindole (DAPI) blue nuclear stain (Invitrogen, Thermo Fisher Scientific, USA).

### Cell Culture

Authenticated IHMGECs were grown and passaged in keratinocyte serum-free medium (KSFM) with 5 ng/mL epidermal growth factor (EGF) and 50 μg/mL bovine pituitary extract (BPE) (Thermo Fisher Scientific, Waltham, Mass.) under 21% O₂ (normoxia) [Liu et al. (2010) Investigative ophthalmology & visual science 51:3993-4005; McDermott et al. (2018) Current eye research 43:1097-1101; Liu et al. (2013) Investigative ophthalmology & visual science 54:2541-2550]. Upon reaching 60% to 70% confluence, culture media were replaced by a 1:1 mixture of Dulbecco's modified Eagle's medium and Ham's F12 (DMEM/F12, Corning Life Sciences, Tewksbury, Mass.), supplemented with 10% fetal bovine serum (FBS, Atlanta Biologicals, Flowery Branch, Ga.) [Sullivan et al. (2014) Investigative ophthalmology & visual science 55:3866-3877; Liu et al. (2014) JAMA ophthalmology 132:226-228]. Cells were cultured for 4-10 days under 21% O₂ (normoxic) or 1% O₂ (hypoxic) conditions. Culture plates of cells in the hypoxic condition were incubated inside hypoxia chambers filled with a premade 1% O₂, 5% CO₂ and 94% N₂ gas mixture (Linde Gas North America, Hammond, Ind.), according to published protocols in Liu et al. (2019) The ocular surface 17(2):310-317 and Wright and Shay (2006) Nat Protoc 1:2088-2090. Following cell medium changes, the chambers were re-flushed with the gas mixture before returning to the incubator. Cells were cultured in the presence or absence of 50 μM Roxa (Selleck Chemical, Houston, Tex.) under normoxic or hypoxic conditions for 4 to 10 days. Cell culture experiments (n=3 wells/treatment) were repeated at least 3 times.

### Western Blot

After culture in DMEM/F12+10% FBS under normoxic conditions for 7 days, cells were exposed to 21% O₂, 1% O₂ and 10 μM Roxa, alone or in combination, for an additional 6 hours. The cells were lysed in 2× Laemmli sample buffer (Bio-Rad Laboratories, Hercules, Calif.) and sonicated to extract nuclear protein [Andersen et al. (2002) Current biology: CB 12:1-11]. Human hepatocellular carcinoma cell lines (HepG2 cells) (America Type Culture Collection, Manassas, Va.), which express high concentration of HIF1α under CoCl₂ stimulation for 4 hours [Yoo et al. (2014) Journal of cellular biochemistry 115:1147-1158], were used as a positive control for HIF1α antibody. The CoCl₂ was purchased from Sigma-Aldrich Corp (St. Louis, Mo.). Cell lysates were then evaluated for HIF1α (1:1000, BD Biosciences, San Jose, Calif.) or β-actin (1:10,000, Cell Signaling Technology) expression. Secondary antibodies were horseradish peroxidase (HRP)-conjugated goat anti-rabbit or goat anti-mouse IgG (both 1:5000, Sigma-Aldrich). Densitometry was performed using ImageJ (at the World Wide Web website of rsb.info.nih.gov/ij).

### Lipid Analyses

After four days of incubation, cells seeded on chamber slides (Corning, Rochester, N.Y.) were stained with 0.3% Oil red 0 (ORO, Fisher Biotec, Wembley, Wash., Australia) according to the published protocol in Liu et al. (2010) Investigative ophthalmology & visual science 51:3993-4005) and Liu et al. (2014) Toxicology 320:1-5. Slides were viewed using a Nikon Eclipse E800 microscope. The size of the stained area was quantified using ImageJ [Deutsch et al. (2014) Anal Biochem 445:87-89].

Cells seeded in 10-cm polystyrene-coated dishes (Corning, Falcon) were extracted with chloroform and methanol, and the lipid extractions were separated by high-performance thin-layer chromatography (HPTLC), as previously described in Liu et al. Toxicology 2014; 320:1-5, Liu and Ding (2014) Investigative ophthalmology & visual science 55:5596-5601, and Liu et al. (2015) Cornea 34:342-346. In brief, for nonpolar lipid analysis, the plate (Silica Gel 60, Merck, Darmstadt, Germany) was developed in hexane: diethyl ether: acetic acid (80:20:1.5, vol/vol/vol) alone. For phospholipids analysis, the plate was developed sequentially in chloroform:ethanol:water (60:30:5 vol/vol/vol) and then hexane:diethyl ether:acetic acid (80:20:1.5, vol/vol/vol) [Liu and Ding (2014) Investigative ophthalmology & visual science 55:5596-5601]. Bands were visualized according to published protocols [Ponec et al. (1988) Journal of lipid research 29:949-961], and the intensities of bands were quantified using ImageJ [Liu and Ding (2014) Investigative ophthalmology & visual science 55:5596-5601].

### Single Radial Enzyme Diffusion Assay (SREDA)

After being treated with 21% O₂, 1% O₂, 10 μM Roxa or a combination for 10 days, cell numbers were counted with a hemocytometer and the cell lysates and cell culture supernatants were collected, as previously described in Liu et al. (2019) The ocular surface 17(2):310-317. DNase II activity in fresh samples was analyzed by SREDA in agarose gels according to published protocols in Liu et al. (2019) The ocular surface 17(2):310-317 and Oka et al. (2012) Nature 485:251-255. Cell lysates and culture media were used to measure the DNase II activity inside and outside of the cells, respectively [Liu et al. (2019) The ocular surface 17(2):310-317]. After incubation for 3-24 hours at 37° C., the area of the dark circle in the gel was measured under an UV trans-illuminator at 312 nm. For comparison between conditions, values were normalized to cell number.

### Statistical Analysis

The significance of the differences between groups was determined by using ANOVA and Fisher's LSD multiple comparisons test (Prism 5, GraphPad Software, Inc., La Jolla, Calif.). Values with p<0.05 were considered statistically significant.

### Results

### Expression of HIF1α in the Human MG

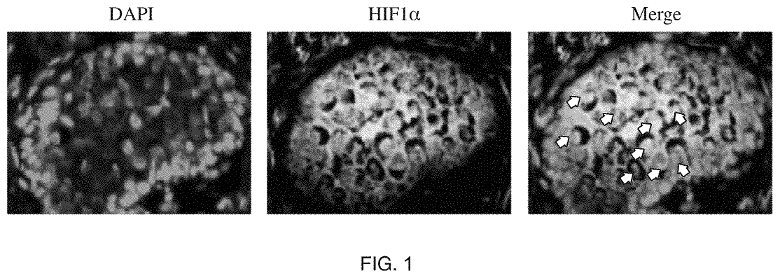

Human eyelid tissues were stained for HIF1α expression to determine whether HIF1α is present in the human MG. As shown in FIG. 1, human MG acinar epithelial cells express HIF1α. This protein appeared to locate primarily within nuclei (arrows), and its expression appeared to be elevated near the central area of acinar complexes.

### Influence of Low O₂ Levels and Roxa on HIF1α Expression in IHMGECs

To determine whether HIF1α is also expressed in vitro, and whether different treatments impact the expression of this protein, IHMGECs were exposed to Roxa (50 μM) in both normoxic (21% O₂) and hypoxic (1% O₂) environments for a 6-hour period.

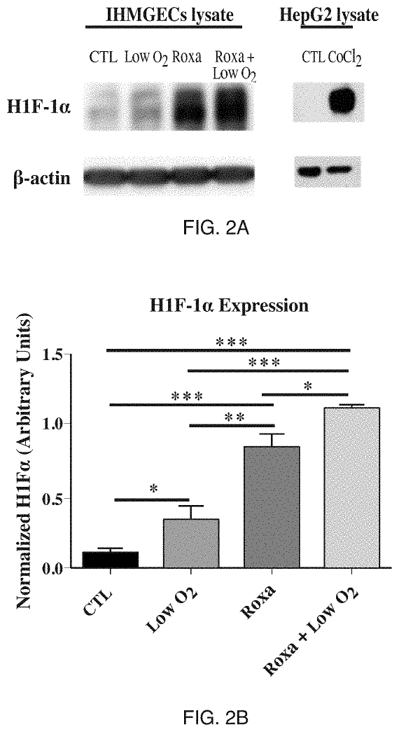

As shown in FIGS. 2A and 2B, the administration of both low O₂ and Roxa significantly increased the HIF1α expression in IHMGECs. Roxa induced significantly more HIF1α expression than low O₂ alone. The combination of both Roxa and low O₂ stimulated the greatest accumulation of HIF1α. As anticipated, the positive control HepG2 cell lysates, harvested after 4 hours of CoCl₂ treatment, expressed substantial amounts of HIF1α protein.

### Impact of Low O₂ and Roxa on Lipid Accumulation and Composition in IHMGECs

To examine whether 1% O₂ exposure and/or Roxa administration stimulate the accumulation of neutral lipids within IHMGECs [Liu et al. (2019) Ocul Surf 17:310-317], IHMGECs were treated with Roxa (50 about 20 μg/ml) in either normoxic (21% O₂) or hypoxic (1% O₂) environments conditions for 4 days.

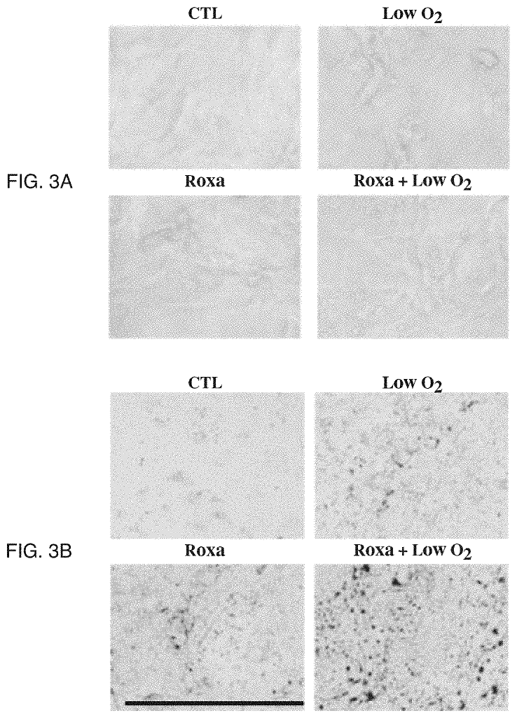

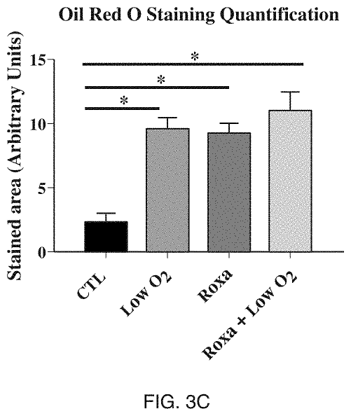

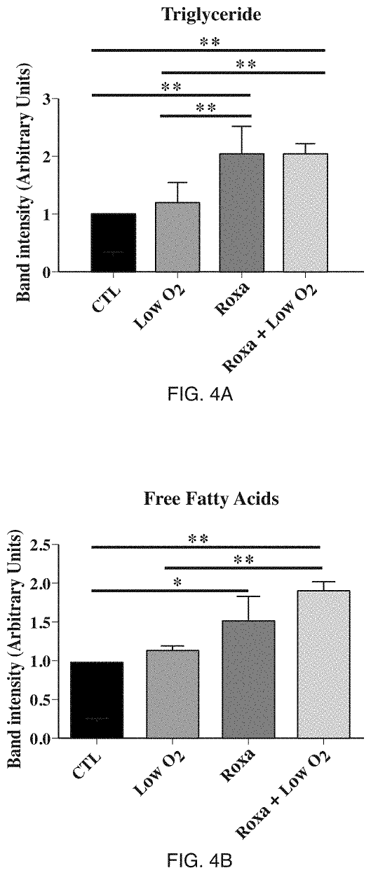

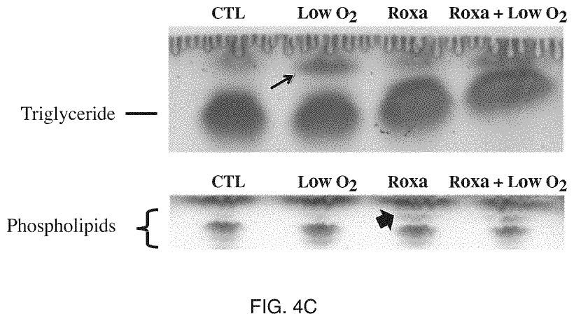

As shown in FIGS. 3A-3C, Roxa duplicated the effect of a hypoxic environment on the accumulation of neutral lipid droplets in IHMGECs, while the combination of Roxa and 1% O₂ environment further increased the amount of lipid droplets. Analysis of nonpolar lipids by HPTLC showed that Roxa, but not low O₂ alone, increased the levels of triglycerides and free fatty acids (FFAs) under either normoxic or hypoxic conditions, as compared to controls (FIGS. 4A-4C). The 1% O₂ and Roxa treatments also appeared to enhance the expression of unidentified nonpolar (the upper panel) and polar lipids (the lower panel), respectively (FIG. 4C). Neither Roxa nor 1% O₂ had any effect on the amounts of cholesterol esters, wax esters, phosphatidylethanolamine, phosphatidylinositol, phosphatidylcholine, phosphatidic acid or lysophosphatidylcholines in IHMGECs.

### Impact of Low O₂ and Roxa on the Terminal Differentiation of IHMGECs

Low O₂ increases DNase II activity and promotes cell terminal differentiation of IHMGECs (Liu et al. Ocul Surf 2019; 17:310-317). To determine whether Roxa treatment can reproduce this hypoxic effect, IHMGECs were treated with Roxa in both normoxic and 1% O₂ conditions for 10 days, prior to counting cell numbers and analyzing DNase II activity in cell lysates and supernatants.

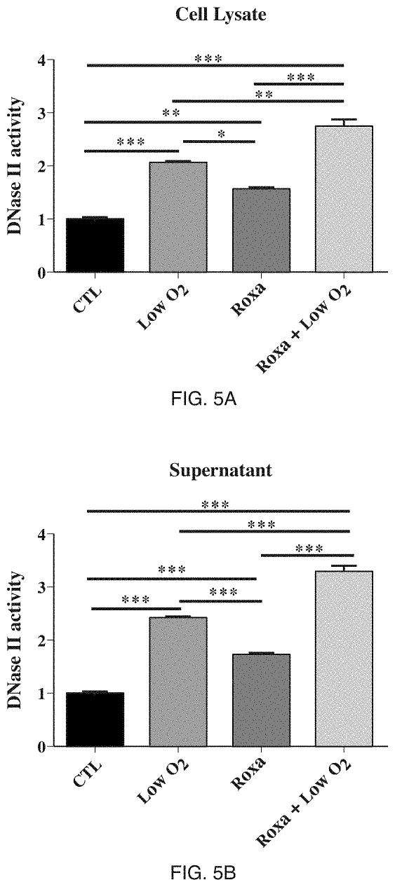

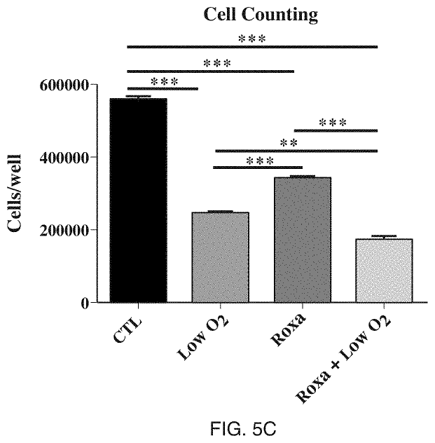

As the result, administration of both Roxa and low O₂ significantly increased the DNase II activity in IHMGEC lysates (FIG. 5A) and culture supernatants (FIG. 5B). The magnitude of this effect was greatest in the Roxa and low O₂ combination, followed by 1% O₂ alone, then by Roxa alone. These effects were paralleled by corresponding significant decreases in cell numbers (FIG. 5C). Such decreases would be expected, given that terminal differentiation of IHMGECs involves cellular disintegration.

### DISCUSSION

It was recently discovered that a hypoxic environment is beneficial for meibomian gland (MG) function. The mechanism(s) underlying this effect was unknown, but may be due to an increase in the levels of hypoxia-inducible factor 1α (HIF1a). In other tissues, HIF1α is the primary regulator of cellular responses to hypoxia and HIF1α expression can be induced by multiple stimuli, including hypoxia and low oxygen mimetic agents. In the current studies described in this Example, human eyelid tissues were stained for HIF1α. Immortalized human MG epithelial cells (IHMGECs) were cultured for varying time periods under normoxic (21% O₂) or hypoxic (1% O₂) conditions, in the presence or absence of the low oxygen mimetic agent Roxadustat (Roxa). IHMGECs were then processed for the analysis of cell number, HIF1α expression, lipid-containing vesicles, neutral and polar lipid content, DNase II activity and intracellular pH. The results show that HIF1α protein is present in human MG acinar epithelial cells in vivo. The findings also demonstrate that exposure to 1% O₂ or to Roxa increases the expression of HIF1α, the number of lipid-containing vesicles, the content of neutral lipids, and the activity of DNase II, and decreases the pH in IHMGECs in vitro. The data support that the beneficial effect of hypoxia on the MG is mediated through an increased expression of HIF1α.

The exemplary experimentation in this Example illustrate that HIF1α mediates the beneficial effects of hypoxia on HMGEC function. While HIF1α is expressed by human MG acinar epithelial cells in vivo, exposure to 1% O₂ or to Roxa increased the levels of HIF1α, the number of lipid-containing vesicles, the content of neutral lipids, and the activity of DNase II in IHMGECs in vitro. These data support an important role for HIFα in the regulation of MG function.

HIF1α proteins are primarily located within nuclei of HMGECs, and especially in those cells situated near the central areas of MG acini. This nuclear location is consistent with findings in other sebaceous glands [Rosenberger et al. (2007) The Journal of Investigative Dermatology 127:2445-2452; Revenco et al. (2017) Stem Cells 35:1355-1364] but uncommon in non-sebaceous tissues [Talks et al. (2000) The American Journal of Pathology 157:411-421]. The accumulation of HIF1α in the center of acinar complexes may be because this area is likely the most hypoxic in the MG. The reason is that the O₂ source for the MG is the vasculature located beyond the MG basement membrane [Knop et al. (2011) Investigative Ophthalmology & Visual Science 52:1938-1978]. By Krogh's law, this would establish an O₂ gradient, with the highest O₂ concentration within the blood vessels and the lowest in the central acinar region. Such hypoxic conditions would be expected to prevent HIF1α degradation and promote its translocation to the nucleus [Bracken et al. (2006) The Journal of Biological Chemistry 281:22575-22585]. The nuclear location of HIF1α in HMGECs indicates that this protein is active and able to stimulate gene transcription [Bracken et al. (2006); Wenger and Gassmann (1997) Biol Chem 378:609-616].

In this Example, exposure to hypoxic conditions, as well as to the low oxygen mimetic Roxa, were found to increase the levels of HIF1α in IHMGECs. The mechanisms underlying these low O₂ and drug responses may be the same. Under normoxic conditions, HIF-prolyl hydroxylase (PH) domain enzymes destabilize HIF proteins by hydroxylating two prolyl residues in the alpha (a) subunit [Yang et al. (2014) Hypoxia (Auckl) 2:127-142]. This HIF1α hydroxylation enables its association with the von Hippel-Lindau tumor suppressor pVHL E3 ligase complex, and leads to the degradation of HIF1α via the ubiquitin-proteasome pathway [Yang et al. (2014)]. However, both low O₂ levels and Roxa inhibit HIF-PH, thereby enhancing the steady-state levels of HIF1α [Bracken et al. (2006)]. Roxa treatment was more effective than hypoxia alone in stimulating the accumulation of HIF1α. This result may be due to a stronger HIF-PH inhibiting effect, or to a delay in achieving the desired O₂ concentration in the culture media during the 6-hour time course of our experiments. O₂ has a relatively low solubility and diffusion rate in aqueous solutions, and its concentration in culture media may not reach equilibrium with the hypoxic chamber air for up to three hours [Allen et al. (2001)Am J Physiol Lung Cell Mol Physiol 281:L1021-1027]. Thus, the IHMGECs may have been exposed to a final 1% O₂ environment, as compared to Roxa, for less time. The combination of both low O₂ and Roxa induced the highest levels of HIF1α.

The 1% O₂ environment and Roxa administration led to a significant accumulation of neutral lipid-containing droplets in IHMGECs. This effect may have been mediated through the increased content of HIF1α, given that this protein is involved in modulating lipid signaling and synthesis, lipid uptake and transport, FFA and sterol metabolism, and lipid droplet biogenesis [Bensaad et al. (2014) Cell Rep 9:349-365; Mylonis et al. (2012) Journal of Cell Science 125:3485-3493; Liu et al. (2014) Toxicology Letters 226:117-123]. These droplets are the major lipid storage organelle in most eukaryotic cells, and contain variable amounts of neutral lipids, including triglycerides and free and esterified sterols, enclosed by a phospholipid monolayer [Olzmann and Carvalho (2019) Nature Reviews Molecular Cell Biology 20:137-155]. The enhanced lipid droplet content in IHMGECs in response to low O₂ and/or Roxa exposure is consistent with the effect of a hypoxic environment in other cell types [Mylonis et al. (2012); Gordon et al. (1977) The American Journal of Pathology 88:663-678; Taniguchi et al. (2013) Nature Medicine 19:1325-1330].

A change in the amount of neutral lipids in response to low O₂ in IHMGECs was not found in a previous study [Liu et al. (2019) The Ocular Surface 17(2):310-317]. These inconsistent findings may be due to several factors, including differences in experimental design and staining procedures. In the previous study, IHMGECs were exposed to 3% O₂, different from the 1% O₂ in this Example. One concern for an exposure to a low O₂ environment is that systemic and prolonged hypoxia may lead to a range of severe pathophysiological reactions and compensations in vivo and would hinder its translation into clinical treatment in the future. However, the current Example utilized an even lower O₂ concentration (1% O₂) because mouse MGs were reported to stain positively for pimonidazole [Liu et al. (2019) The Ocular Surface 17(2):310-317], indicating that the oxygen level in MGs is less than 1.3% [Gross et al. (1995) International Journal of Cancer Journal international du cancer 61:567-573]. In addition, in the previous study, LipidTOX reagent was used to identify neutral lipids in IHMGECs, detecting most staining occurred within lysosomes [Sullivan et al. (2014) Investigative ophthalmology & visual science 55:3866-3877; Liu et al. (2014) Toxicology 320:1-5]. These lysosomes may well be lamellar bodies, which contain cholesterol, neutral lipids, phospholipids and various enzymes [Liu et al. (2014) Toxicology 320:1-5; Schmitz and Muller (1991) Journal of Lipid Research 32:1539-1570; Feingold (2007) Journal of Lipid Research 48:2531-2546]. In this Example, ORO and bright-field microscopy permitted the visualization of neutral lipids in lipid droplets [Mehlem et al. (2013) Nat Protoc 8:1149-1154; Fam et al. (2018) Materials (Basel) 11:1768]. Both lamellar bodies and lipid droplets are organelles specialized for lipid storage, and there is evidence that lamellar bodies may be derived from, or transform into, lipid droplets [Menon et al. (2018) Journal of Dermatological Science 92:10-17]. Considering that both lipid droplets and lamellar bodies exist in the human MG [Gorgas and Volkl (1984) The Histochemical Journal 16:1079-1098; Jester et al. (1981) Investigative Ophthalmology & Visual Science 20:537-547], their specific roles in lipid dynamics await clarification. Lastly, different methods were used to quantitate the extent of neutral lipid expression in the previous study versus this Example. For the former the intensity of LipidTOX staining was measured, whereas in this Example the area of ORO staining was calculated. Intensity and area measurements do not necessarily lead to equivalent results.

The administration of 1% O₂ and/or Roxa promoted the terminal differentiation of IHMGECs, as indicated by the significant increase in DNase II activity in cell lysates and culture supernatants. DNase II is a lysosomal enzyme [Ohkouchi et al. (2013) PloS one 8:e59148], which is typically activated by acidic conditions (pH 4.5-5.5) and then translocates from lysosomes to the nucleus [Evans and Aguilera (2003) Gene 322:1-15]. Within that location DNase II triggers programmed cell death and ultimately holocrine secretion [Fischer et al. (2017) The Journal of Investigative Dermatology 137:587-594; Barry and Eastman (1993) Arch Biochem Biophys 300:440-450]. The heightened levels of HIF1α may have mediated this process. Activation of HIF1α stimulates glycolysis and lactic acid production, which decreases the intracellular pH [Brahimi-Horn and Pouyssegur (2007) Essays Biochem 43:165-178]. HIF1α also reduces the intracellular pH through modulation of carbonic anhydrase (CA) IX [Logsdon et al. (2016) Molecular Cancer Therapeutics 15:2722-2732]. CA IX expression, recently identified in HMGECs in vivo, is increased by HIF1α [Kaluz et al. (2009) Biochimica et biophysica acta 1795:162-172]. The CA enzymes, in turn, could increase intracellular H+ mobility and promote cellular acidification [Boron (2004) Adv Physiol Educ 28:160-179]. These HIF1α effects on pH would destabilize lysosomal membranes, cause the release of lysosomal enzymes, and lead to the activation of DNase II [Tapper and Sundler (1990) The Biochemical Journal 272:407-414; Sundler (1997) Acta Physiol Scand 161:553-556; Torriglia et al. (1998) Molecular and Cellular Biology 18:3612-3619; Eastman (1994) Cell Death and Differentiation 1:7-9]. Hypoxic environments and mimetics also induce such lysosomal disruption in other cell types [Eastman (1994); Smith et al. (1974) The Journal of pharmacology and experimental therapeutics 191:564-574; Abraham et al. (1967) Nature 215:194-196; Loegering et al. (1975) Experimental and Molecular Pathology 22:242-251].

Overall, an important role for HIF1α in the regulation of MG function is illustrated in this Example. Local administration of hypoxia mimetics may be beneficial for the treatment of MGD.

### Example 2

### Roxadustat (Roxa) Treatment for Meibomian Gland Dysfunction (MGD) In Vivo

After the discovery that the oxygen-sensitive protein HIF1α plays an essential role in the regulation of human meibomian gland epithelial cells (HMGECs), alternative treatments for MGD and other related diseases or disorders have been tested, aiming to avoid any pathophysiological reactions and compensations caused by the systemic and prolonged hypoxia treatment. In addition to a hypoxic environment, HIF1α expression also can be induced by low oxygen mimetic agents and growth factors. In order to mimic a local hypoxic environment around the MG, but also to minimize the compensatory effects of systemic low oxygen exposure, pharmacological activation of the HIF1α pathway under normoxic (21% O₂) conditions were tested. The hypoxia mimetic Roxadustat (Roxa; also called FG-4592) duplicated the differentiation-promoting effect of low oxygen by promoting HIF1α in immortalized HMGECs under normoxic condition. Roxa has previously been tested for safety in animal studies and has passed a phase III clinical trial for the treatment of anemia in patients with chronic kidney disease. Thus, Roxa may be a safety drug to use in the current studies.

Animal studies were used to determine the capacity of Roxa to ameliorate MGD in an model of apolipoprotein E-deficient (ApoE −/−) mice, which have recently been described as a model for MGD. These knockout mice have disrupted lipid regulation (i.e. cholesterol and heart disease) and are widely used as a model of obesity with accelerated atherosclerosis.

Specifically, two groups of ApoE KO (ApoE −/−) mice, comprising eight (8) male mice for each group, were treated with either vehicle control or Roxa. In addition, four (4) age-matched wild-type C57BL/6J male mice were untreated, as normal meibomian gland controls.

At the beginning of the study, the 16 ApoE−/− mice and 4 wild-type (WT) C57BL/6J mice, 12 weeks of age, were measured for their weight, tear volume, corneal fluorescein staining and tear breakup time (TBUT). The ApoE −/− mice were randomly divide into 2 groups (n=8 mice/group) and treated with vehicle control (2% DMSO in sterile saline, subcutaneous injection) or Roxa (10 mg/kg, subcutaneous injection). The injections were administrated three times per week (on Monday, Wednesday, and Friday mornings) for 12 weeks. The ApoE −/− mice were weighted weekly (on Mondays) to determine Roxa injection volume for each individual. At the end of the study, the weight, tear volume, corneal fluorescein staining and tear breakup time (TBUT) were measured again. The eyelids of each mouse were then dissected to assess meibomian gland size and morphology.

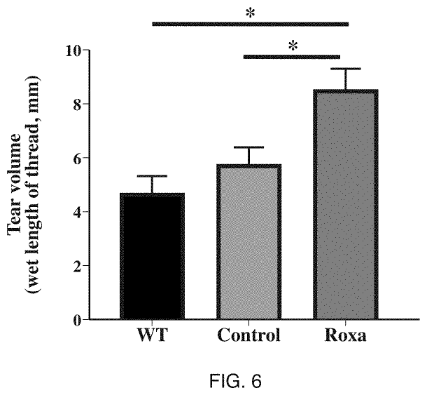

As shown in FIG. 6, Roxa treatment (“Roxa”) significantly increased the tear volume in the mice comparing to the control group (“Control”) and the wild type (“WT”) mice (* p<0.01). Tear volume is generally recognized as an important criterion in evaluating MG and dry eye disease.

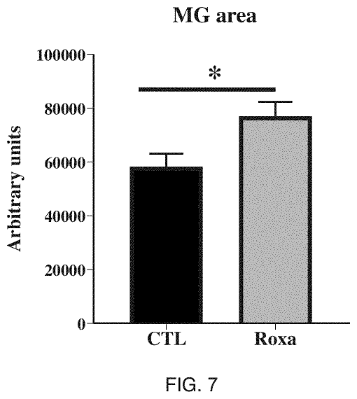

As shown in FIG. 7, Roxa treatment also significantly reduced the meibomian gland loss in the mice eyelids (“Roxa”) comparing to the control group (“CTL”) (* p<0.05). Meibomian gland dysfunction is usually accompanied with gland dropout. Once happened, no previously known methods may reverse or alleviate the dropouts. The effect of Roxa shown here represents the first solution to reduce meibomian gland dropout and reserve the MG function.

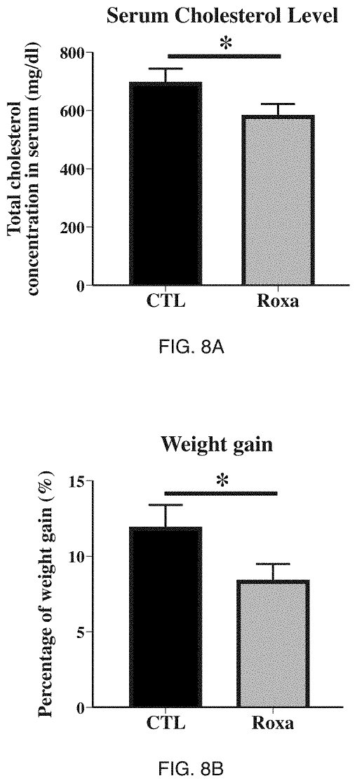

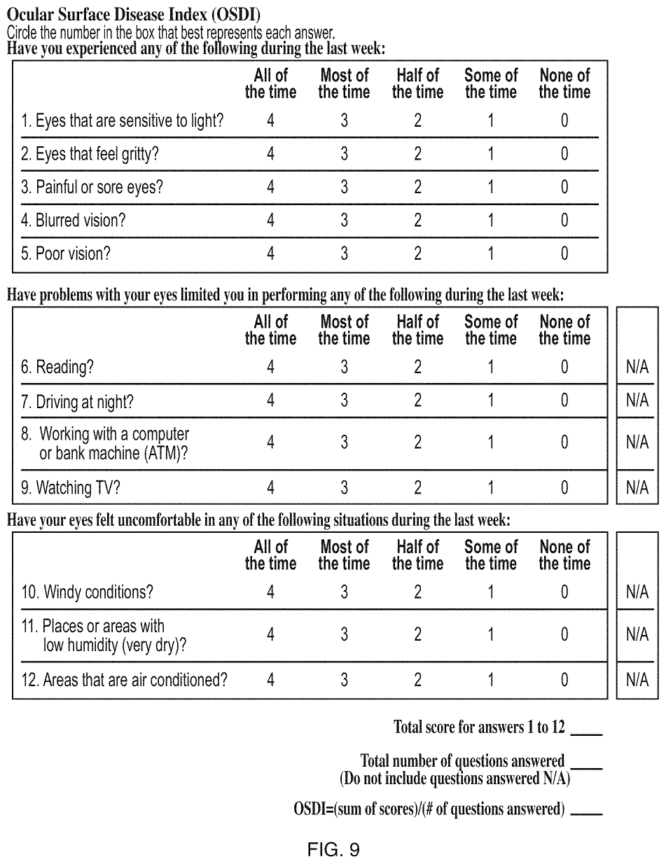

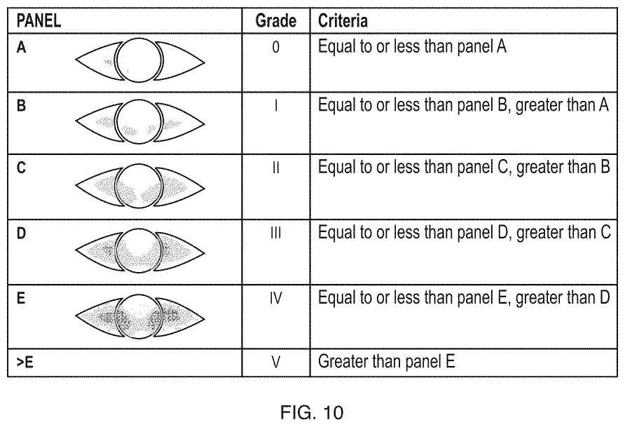

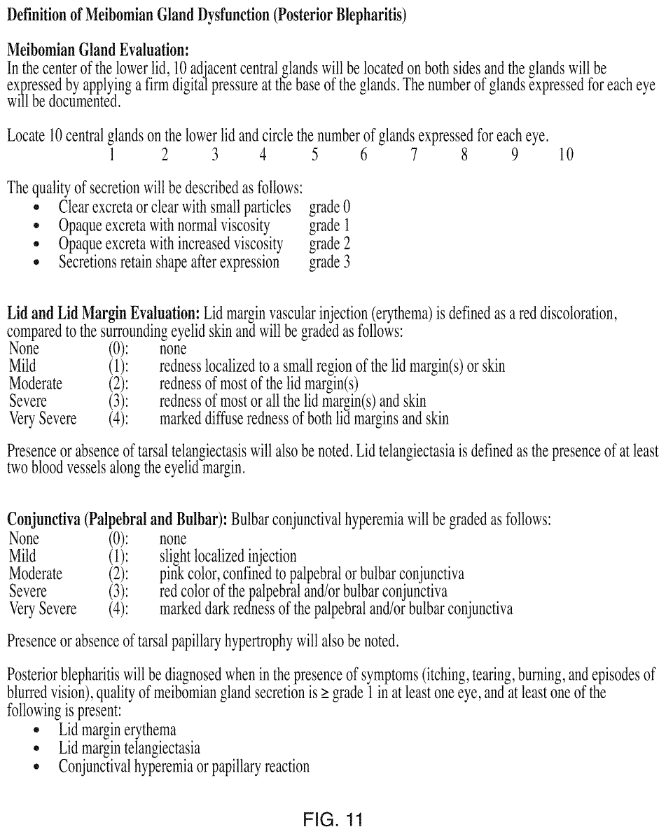

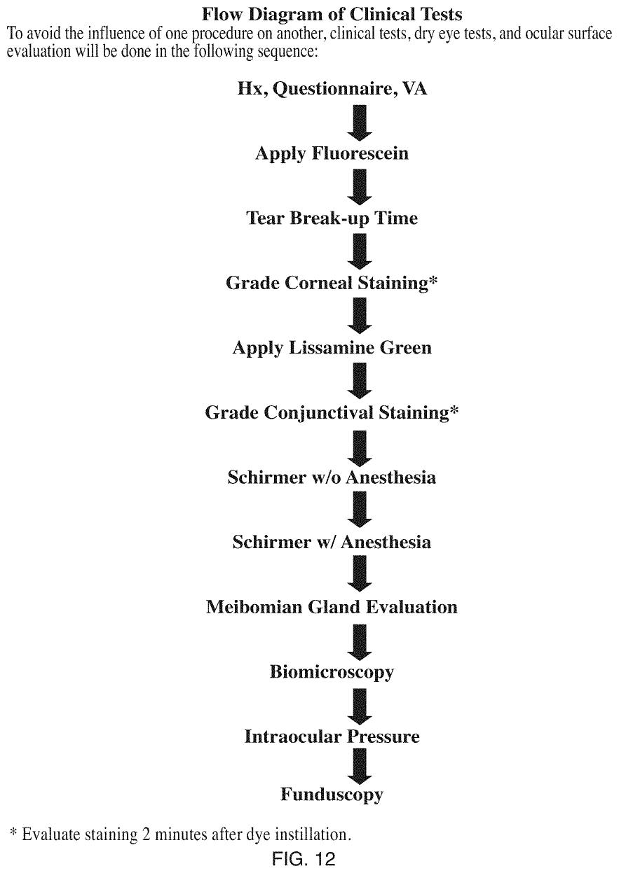

Besides the changes in the eye, Roxa treatment also significantly reduced the serum cholesterol level (FIG. 8A) and the percentage of weight gain (FIG. 8B) in the mice (* p<0.05). This result indicates that Roxa has a potential weight control effect to treat obesity.

The mice eyelids were collected and dissected after sacrificing. The dissected samples were stained with different antibodies and dyes for lipids. For example, the dissected mice lid samples were stained with Oil Red O and LipidTox™ (Invitrogen) for evaluation of lipid accumulation. The sections were stained for pimonidazole, DNase II, HIF1α, PPARγ, Desmoglein and Desmocollin for the evaluation of cell differentiation and maturation. Roxa treatment promoted expression of the several key markers for MG cell differentiation in mice. These markers include intracellular lipid accumulation, as well as expression of peroxisome proliferator-activated receptor gamma (PPAR-γ), Desmoglein and Desmocollin in MG cells. These results further indicate that Roxa treatment may be used to treat obesity or other diseases or disorders characterized with increased lipid accumulation.

The results from the studies suggest that Roxa can cure or at least alleviate MGD and DED. A clinical study will be planned for further testing Roxa drug development. Roxa may be developed into a topically applicable eye drop or eye ointment for a phase I clinical trial.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications and sequence (nucleic acid or protein) citations cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of reducing the severity or treating an optical disease or disorder associated with reduced meibomian gland (MG) function in a subject by locally administering to an eye tissue of the subject a composition comprising a low oxygen mimetic compound, wherein reduced severity or treatment of said disease or disorder comprises increased tear secretion, increased tear volume, prolonged tear film break up time, improved tear osmolarity, improved gland expressibility, improved gland expressed oil quality and volume, increased thickness of tear film lipid layer, decreased ocular surface staining, decreased inflammation on the ocular surface, or alleviated ocular discomfort feeling of patients.

2. The method of claim 1, wherein said compound comprises a hypoxia inducible factor (HIF) prolyl hydroxlase inhibitor (HIF-PHI).

3. The method of claim 1, wherein said low oxygen mimetic compound comprises Roxadustat, Daprodustat, Molidustat, Vadadustat, or Desitustat.

4. The method of claim 1, wherein said composition in the form of an eye drop or eye ointment.

5. The method of claim 1, wherein said composition is administered at a concentration of 0.01-100 mg/ml.

6. The method of claim 1, wherein said compound is administered at a concentration of 1-50 μg/ml.

7. The method of claim 1, wherein said compound is administered to an eye tissue by injection.

8. The method of claim 1, wherein said compound is administered by subconjunctival injection, subdermal injection around an eyelid, or periorbital injection.

9. The method of claim 6, wherein said compound is administered at a dose of 1-300 μg per injection.

10. A method of reducing the severity or treating an optical disease or disorder associated with reduced meibomian gland (MG) function in a subject by systemically administering to the subject a composition comprising a low oxygen mimetic compound, wherein reduced severity or treatment of said disease or disorder comprises increased tear secretion, increased tear volume, prolonged tear film break up time, improved tear osmolarity, improved gland expressibility, improved gland expressed oil quality and volume, increased thickness of tear film lipid layer, decreased ocular surface staining, decreased inflammation on the ocular surface, or alleviated ocular discomfort feeling of patients.

11. The method of claim 10, wherein said compound is administered orally at a dose of 50-70 mg per oral administration or 1-10 mg/kg of body weight per injection.

12. The method of claim 10, wherein the oral dose does not exceed 3.0 mg/kg of body weight.

13. The method of claim 1 or 10, wherein local oxygen concentration at the MG in the subject is reduced to no more than 1%.

14. The method of claim 1 or 10, wherein the agent in iii) comprises Roxadustat (Roxa), dimethyloxalyglycine, desferrioxamine, cobalt (II) chloride (CoCl₂), of combinations thereof.

15. A method of promoting differentiation of a meibomian gland epithelial cell (MGEC) by
i) reducing local oxygen concentration of the MGEC to no more than 1%; and/or
ii) contacting the MGEC with an HIP-PHI.

16. The method of claim 15, wherein the agent comprises roxadustat (Roxa), dimethyloxalyglycine, desferrioxamine, cobalt (II) chloride (CoCl₂), FG-2216, daprodustat/GSK1278863, vadadustat/AKB-6548, molidustat/BAY 85-3934, desidustat/ZYAN1), Dimethyloxalylglycine (DMOG), or combinations thereof.

17. A method of treating obesity and/or reducing weight gain of a subject by administering to the subject a pharmaceutically effective dosage of an HIF-PHI agent capable of reducing oxygen concentration, wherein said subject comprises obesity with high serum cholesterol level.

18. The method of claim 17, wherein the agent comprises Roxadustat (Roxa), dimethyloxalyglycine, desferrioxamine, cobalt (II) chloride (CoCl₂), of combinations thereof.

19. A dosage formulation for administration to a subject, wherein the formulation comprises Roxadustat (Roxa)
i) in a therapeutically effective amount between about 0.1-100 mg/ml for administration as an eye drop for treatment of reduced meibomian gland (MG) function; or
ii) in a therapeutically effective amount between about 1-300 μg for administration as an injection for treatment of reduced meibomian gland (MG) function.

20. A dosage formulation for administration to a subject, wherein the formulation comprises Roxadustat (Roxa) in a therapeutically effective amount between about 50-500 mg for treatment of obesity and/or elevated serum cholesterol, wherein said effective amount is administered 1-5 times per week.
